(19) United States
(12) Patent Application Publication
(10) Pub. No.: US 2026/0148860 A1
(43) Pub. Date: May 28, 2026

(54) DYNAMICAL SYSTEM MODELING FOR IMMUNE STABILITY ANALYSIS

(71) Applicant: City of Hope, Duarte, CA (US)

(72) Inventors: Russell Rockne, Duarte, CA (US); Flavia Pichiorri, Duarte, CA (US); Amrita Krishnan, Duarte, CA (US); Vikram Adhikarla, Duarte, CA (US); Alexander Brummer, Duarte, CA (US); Mahmoud Singer, Duarte, CA (US); Ada Dona, Duarte, CA (US); Ryan Woodall, Duarte, CA (US)

(21) Appl. No.: 19/402,810

(22) Filed: Nov. 26, 2025

Related U.S. Application Data
(60) Provisional application No. 63/726,074, filed on Nov. 27, 2024.

(51) Int. Cl. G16H 50/50

(52) U.S. Cl. CPC G16H 50/50

(57) ABSTRACT

A device may generate, based at least on time series data of a quantity of one or more markers present in a blood sample of a subject, a dynamical system model representative of an immune system of the subject, the dynamical system model including one or more functions corresponding to a derivative of the time series data, and the generating of the dynamical system model includes performing a regression to identify, within a library of candidate functions, one or more functions for inclusion in the dynamical system model. A device may determine a steady state solution for the one or more functions included in the dynamical system model. A device may determine, based at least on the steady state solution for the one or more functions included in the dynamical system model, a stability of the immune system of the subject. Related systems and computer program products are also provided.

### CROSS REFERENCE TO RELATED APPLICATIONS

This application claims priority to, and benefit of, U.S. Provisional Patent Application No. 63/726,074, filed Nov. 27, 2024, the contents of which are incorporated by reference in their entirety.

### TECHNICAL FIELD

The subject matter described herein relates generally to dynamical systems and more specifically to dynamical system modeling for characterizing the state of an immune system.

### BACKGROUND

Cancer arises when normal cells acquire genetic mutations that enable uncontrolled proliferation and evasion of immune surveillance. Under healthy conditions, the immune system plays a critical role in identifying and eliminating abnormal cells, thereby maintaining tissue integrity and preventing malignancy. This process relies on a complex network of immune cell subsets functioning in concert to provide effective surveillance.

However, many cancers develop mechanisms to circumvent immune detection. Tumor cells frequently exploit immune-evasion strategies, such as downregulating antigen presentation or inducing immunosuppressive signaling, which can weaken or co-opt immune populations. These disruptions often lead to imbalances in immune cell subsets, impairing the body's ability to mount an effective anti-tumor response.

Consequently, longitudinal monitoring of immune system dynamics is essential for early detection of disease progression, guiding therapeutic interventions, and managing long-term outcomes. Traditional approaches relying on static biomarkers fail to capture the evolving nature of immune responses, underscoring the need for dynamic, systems-based methods to characterize immune stability and predict treatment efficacy.

### SUMMARY

Systems, methods, and articles of manufacture, including computer program products, are provided for immune stability and circulating plasma cell analysis.

In some aspects, the techniques described herein relate to a computer-implemented method, including: generating, based at least on time series data of a quantity of one or more markers present in a blood sample of a subject, a dynamical system model representative of an immune system of the subject, the dynamical system model including one or more functions corresponding to a derivative of the time series data, and the generating of the dynamical system model includes performing a regression to identify, within a library of candidate functions, the one or more functions for inclusion in the dynamical system model; determining a steady state solution for the one or more functions included in the dynamical system model; and determining, based at least on the steady state solution for the one or more functions included in the dynamical system model, a stability of the immune system of the subject.

In some aspects, the techniques described herein relate to a method, wherein the subject is a patient with multiple myeloma.

In some aspects, the techniques described herein relate to a method, wherein the subject is a healthy individual.

In some aspects, the techniques described herein relate to a method, wherein the subject is a patient with an immune mediated cancer.

In some aspects, the techniques described herein relate to a method, wherein the regression is a sparse regression.

In some aspects, the techniques described herein relate to a method, wherein the time series data is derived from at least one of data from blood analysis, the blood analysis including: complete blood count, differential white blood cell count, spectral flow cytometry (Cytek), or Mass cytometry (CyTOF).

In some aspects, the techniques described herein relate to a method, wherein the sparse regression is performed to determine a sparse vector of coefficients indicating the one or more functions as being active.

In some aspects, the techniques described herein relate to a method, wherein the library of candidate functions include one or more constant functions, polynomial functions, and trigonometric functions.

In some aspects, the techniques described herein relate to a method, wherein the one or more functions include nonlinear functions.

In some aspects, the techniques described herein relate to a method, wherein the steady state solution of the one or more functions included in the dynamical system model includes one or more eigenvalues.

In some aspects, the techniques described herein relate to a method, wherein the immune system of the subject is determined to be stable based at least on the one or more eigenvalues not exceeding a threshold value, and wherein the immune system of the subject is determined to be unstable based at least on the one or more eigenvalues exceeding the threshold value.

In some aspects, the techniques described herein relate to a method, wherein the threshold value is zero.

In some aspects, the techniques described herein relate to a method, wherein the derivative of the time series data includes a numerical approximation.

In some aspects, the techniques described herein relate to a method, wherein the derivative of the time series data is measured.

In some aspects, the techniques described herein relate to a method, wherein the time series data of the quantity of one or more markers present in the blood sample of the subject includes a quantity of peripheral blood mononuclear (PBMCs) present in the blood sample, or quantity of materials released by tumor cells (e.g., cytokines, transcription factors, DNA).

In some aspects, the techniques described herein relate to a method, further including: performing, on the blood sample of the subject, a cytometry by time of flight (CyTOF) to determine the quantity of the one or more markers present therein.

In some aspects, the techniques described herein relate to a method, further including: performing, on the blood sample of the subject, spectral cytometry (CyTEK) to determine the quantity of the one or more markers present therein.

In some aspects, the techniques described herein relate to a method, wherein one or more antibodies are used to determine the quantity of one or more markers present in the blood sample, the one or more antibodies including ANTI-FITC (CD38 multi-epitope), CD86/B7.2, CD45, CD80/B7-1, CD127/IL.7Ra, CD27, CD3, CD19, CD69, CD138/Syndecan 1, CD25, CD64, CD33, GRANZYME B, CD15/SSEA-1, CD8, HLA-DR, CD45RA, CD279/PD-1, CD14, CD56, CD223/LAG-3, TIGIT, CD269 (BCMA), CD319 (CS-1), gammadelta2 (TCR Vdelta2), CD163, TIM-3, CD16, CD4, CD274/PDL1, CD197/CCR7, CD11b, IgG-Kappa, IgG-Lambda, M-protein, and/or CD20.

In some aspects, the techniques described herein relate to a method, further including: determining, based at least on the stability of the immune system of the subject or specific cellular interaction terms identified in the matrix, one or more of (i) a first likelihood of the subject responding to a treatment, (ii) a second likelihood of the subject relapsing after the treatment, and (iii) a durability of the subject's response to the treatment.

In some aspects, the techniques described herein relate to a method, wherein the treatment includes a therapy affecting the immune system.

In some aspects, the techniques described herein relate to a method, wherein the treatment includes a CD38 antibody (e.g., Daratumumab).

In some aspects, the techniques described herein relate to a method, wherein the treatment includes Chimeric Antigen Receptor (CAR) T-cell therapy.

In some aspects, the techniques described herein relate to a method, further including: identifying, based at least on the stability of the immune system of the subject, one or more treatments affecting the immune system of the subject.

In some aspects, the techniques described herein relate to a method, further including: determining, based at least on the stability of the immune system of the subject, one or more of a dosage and a timing of an immune modulating treatment for the subject.

In some aspects, the techniques described herein relate to a method, wherein the dynamical system model of the immune system of the subject is further determined based at least on a quantity of circulating myeloma plasma cells present in the blood sample of the subject.

In some aspects, the techniques described herein relate to a method, further including: determining, based at least on the quantity of circulating myeloma plasma cells present in the blood sample of the subject, a level of minimal residual disease for the subject.

In some aspects, the techniques described herein relate to a method, further including: determining, based at least on the stability of the immune system of the subject, a level of minimal residual disease for the subject.

In some aspects, the techniques described herein relate to a method, wherein the dynamical system model representative of the immune system of the subject is further generated based time series data of a level of minimal residual disease present in the subject with an immune-mediated cancer.

In some aspects, the techniques described herein relate to a method, wherein generating of the dynamical system model includes filtering the library of candidate functions to exclude functions within the library of candidate functions having an oscillation frequency exceeding two times a maximum sampling frequency of the data.

In some aspects, the techniques described herein relate to a method, further including: reducing a dimensionality of the time series data by applying principal components analysis.

In some aspects, the techniques described herein relate to a system, including: at least one data processor; and at least one memory storing instructions, which when executed by the at least one data processor, result in operations including: generating, based at least on time series data of a quantity of one or more markers present in a blood sample of a subject, a dynamical system model representative of an immune system of the subject, the dynamical system model including one or more functions corresponding to a derivative of the time series data, and the generating of the dynamical system model includes performing a regression to identify, within a library of candidate functions, the one or more functions for inclusion in the dynamical system model; determining a steady state solution for the one or more functions included in the dynamical system model; and determining, based at least on the steady state solution for the one or more functions included in the dynamical system model, a stability of the immune system of the subject.

In some aspects, the techniques described herein relate to a non-transitory computer readable medium storing instructions, which when executed by at least one data processor, result in operations including: generating, based at least on time series data of a quantity of one or more markers present in a blood sample of a subject, a dynamical system model representative of an immune system of the subject, the dynamical system model including one or more functions corresponding to a derivative of the time series data, and the generating of the dynamical system model includes performing a regression to identify, within a library of candidate functions, the one or more functions for inclusion in the dynamical system model; determining a steady state solution for the one or more functions included in the dynamical system model; and determining, based at least on the steady state solution for the one or more functions included in the dynamical system model, a stability of the immune system of the subject.

In some aspects, the techniques described herein relate to a computer-implemented method, including: generating, based at least on time series data of a level of minimal residual disease (MRD) present in a subject with a cancer, a dynamical system model representative of an immune system of the subject, the dynamical system model including one or more functions corresponding to a derivative of the time series data, and the generating of the dynamical system model includes performing a regression to identify, within a library of candidate functions, the one or more functions for inclusion in the dynamical system model; determining a steady state solution for the one or more functions included in the dynamical system model; and determining, based at least on the steady state solution for the one or more functions included in the dynamical system model, a stability of the immune system of the subject.

In some aspects, the techniques described herein relate to a method, wherein the malignancy or cancer is multiple myeloma.

In some aspects, the techniques described herein relate to a system, including: at least one data processor; and at least one memory storing instructions, which when executed by the at least one data processor, result in operations including: generating, based at least on time series data of a level of minimal residual disease (MRD) present in a subject with a cancer, a dynamical system model representative of an immune system of the subject, the dynamical system model including one or more functions corresponding to a derivative of the time series data, and the generating of the dynamical system model includes performing a regression to identify, within a library of candidate functions, the one or more functions for inclusion in the dynamical system model; determining a steady state solution for the one or more functions included in the dynamical system model; and determining, based at least on the steady state solution for the one or more functions included in the dynamical system model, a stability of the immune system of the subject.

In some aspects, the techniques described herein relate to a system, wherein the cancer may be multiple myeloma, liquid tumors, or other cancers.

In some aspects, the techniques described herein relate to a non-transitory computer readable medium storing instructions, which when executed by at least one data processor, result in operations including: generating, based at least on time series data of a level of minimal residual disease (MRD) present in a subject with a blood-borne cancer (e.g., lymphoma, myeloma, leukemia), a dynamical system model representative of an immune system of the subject, the dynamical system model including one or more functions corresponding to a derivative of the time series data, and the generating of the dynamical system model includes performing a regression to identify, within a library of candidate functions, the one or more functions for inclusion in the dynamical system model; determining a steady state solution for the one or more functions included in the dynamical system model; and determining, based at least on the steady state solution for the one or more functions included in the dynamical system model, a stability of the immune system of the subject.

In some aspects, the techniques described herein relate to a non-transitory computer readable medium, wherein the cancer is multiple myeloma.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to immune stability analysis in the context of cancer or multiple myeloma, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a machine learning enabled treatment analysis system, in accordance with some example embodiments;

FIG. 2 depicts graphs illustrating an immune stability analysis for two example patients, in accordance with some example embodiments;

FIG. 3A depicts graphs illustrating the dynamical system models of the immune systems of three additional example patients, in accordance with some example embodiments;

FIG. 3B depicts graphs illustrating plotting the eigenvalues forming the steady state solutions for the dynamical system models of the immune systems of the three additional example patients, in accordance with some example embodiments;

FIG. 4 depicts a flowchart illustrating an example of a process for immune stability analysis, in accordance with some example embodiments;

FIG. 5 depicts a block diagram illustrating an example of a computing system, in accordance with some example embodiments;

FIG. 6 depicts experimental results produced by the dynamical system models of the immune systems of eleven example patients, in accordance with some example embodiments;

FIG. 7 depicts validation results for the dynamical system models of the immune systems as applied to three of the eleven example patients, in accordance with some example embodiments;

FIG. 8 depicts results for the dynamical system models of the immune systems as applied to the eleven example patients, in accordance with some example embodiments;

FIG. 9 depicts principal components analysis as used for data dimensionality reduction in connection with the dynamical system models of the immune systems as applied to eleven example patients, in accordance with some example embodiments;

FIG. 10 depicts data associated with the principal components of patients determined to have stable immune systems in connection with the dynamical system models of the immune systems as applied to eleven example patients, in accordance with some example embodiments;

FIG. 11 depicts data associated with the principal components of patients determined to have un-stable immune systems while presenting as clinically stable in connection with the dynamical system models of the immune systems as applied to eleven example patients, in accordance with some example embodiments;

FIG. 12 depicts data associated with the principal components of patients determined to have unstable immune systems in connection with the dynamical system models of the immune systems as applied to eleven example patients, in accordance with some example embodiments;

FIG. 13 depicts a bar graph associated with the principal components of patients in connection with the dynamical system models of the immune systems as applied to eleven example patients, in accordance with some example embodiments;

FIG. 14 depicts results for the dynamical system models of the immune systems as applied to eighteen example patients, in accordance with some example embodiments;

FIG. 15 depicts results for the dynamical system models of the immune systems as applied to the eighteen example patients and the corresponding impact of dara on various cell populations, in accordance with some example embodiments;

FIG. 16 depicts results for the dynamical system models of the immune systems as applied to the eighteen example patients and the progression of disease in relation to coefficients corelated with immune stability, in accordance with some example embodiments.

FIG. 17 depicts experimental results (e.g., longitudinal expansion kinetics) for the dynamical system models of the immune systems in patients receiving CAR T-cell treatment, in accordance with some example embodiments;

FIG. 18 depicts experimental results (e.g., immune cell clusters) for the dynamical system models of the immune systems in patients receiving CAR T-cell treatment, in accordance with some example embodiments;

FIG. 19 depicts experimental results (e.g., longitudinal remodeling of major immune cell populations) for the dynamical system models of the immune systems in patients receiving CAR T-cell treatment, in accordance with some example embodiments;

FIG. 20 depicts experimental results (e.g., longitudinal dynamics of immune cell subsets) for the dynamical system models of the immune systems in patients receiving CAR T-cell treatment, in accordance with some example embodiments;

FIG. 21 depicts experimental results (e.g., immune cell trajectories for one subject) for the dynamical system models of the immune systems in patients receiving CAR T-cell treatment, in accordance with some example embodiments;

FIG. 22 depicts experimental results (e.g., summary of patient-level modeling outcomes) for the dynamical system models of the immune systems in patients receiving CAR T-cell treatment, in accordance with some example embodiments;

FIG. 23 depicts experimental results (e.g., hierarchical clustering and correlation analysis for immune interactions) for the dynamical system models of the immune systems in patients receiving CAR T-cell treatment, in accordance with some example embodiments; and

FIG. 24 depicts experimental results (e.g., categorical analysis of patient outcomes using binary cutoffs for durable response) for the dynamical system models of the immune systems in patients receiving CAR T-cell treatment, in accordance with some example embodiments.

When practical, similar reference numbers denote similar structures, features, or elements.

### DETAILED DESCRIPTION

The present disclosure relates to the analysis and determination of immune stability of subjects. Subjects may include patients having a cancer (e.g., multiple myeloma) or healthy subjects. In some implementations, immune stability of subjects can be determined and used to determine a treatment plan (e.g., therapeutic agent for use, and/or dosage of a therapeutic agent to be used). In some implementations, the techniques described herein can be performed on non-specific data sets of blood data that are generally available without requiring complex starting blood data, providing enhanced insight into immune stability from blood data that was not previously available.

In some implementations, the systems and methods described herein may be applied to a subject who is a patient having multiple myeloma. Although multiple myeloma is described herein, it is envisioned that the systems and methods described herein may be applicable to other immune mediated cancers such as melanomas, non-small cell lung cancer, renal cancer, bladder cancer, Hodgkin & Non-Hodgkin lymphoma, cervical cancer, head and neck cancer, liver cancer, colorectal cancer, esophageal cancer, leukemia, glioblastomas, breast cancer, prostate cancer and the like.

For example, multiple myeloma is a type of blood cancer affecting a type of white blood cells called plasma cells. Healthy plasma cells produce antibodies as a part of a body's immune response. By contrast, cancerous plasma cells are unable to produce infection fighting antibodies. Instead, cancerous plasma cells produce a variety of abnormal proteins including monoclonal immunoglobulin, monoclonal protein (M-protein), M-spike, and paraprotein. Some complications from multiple myeloma include anemia (e.g., shortage of red blood cells), thrombocytopenia (e.g., shortage of platelets), and leukopenia (e.g., shortage of normal white blood cells), all of which occur when cancerous plasma cells accumulate in the bone marrow and displace normal blood-forming cells. Multiple myeloma may also trigger bone deterioration (e.g., lytic lesions) and reduced kidney function.

Although there is currently no cure for multiple myeloma, the cancer and its various symptoms can be treated, for example, with an immune modulating treatment, to achieve partial or complete remission. Nevertheless, existing criteria for assessing a patient's response to a treatment for multiple myeloma are deficient for a number of reasons. For example, while biomarkers are thought to be helpful in predicting a patient's response to a particular treatment, existing biomarkers are static biomarkers derived from a pre-treatment sample (e.g., tumor tissue, peripheral blood, and/or the like) to capture the state of a complex and evolving biological system at a single point in time. Owing to the dynamical nature of biological systems, such as a patient's immune system, static biomarkers are often poor indicators of how the system's behavior will change over time. Existing static biomarker-based techniques are therefore insufficient for determining a patient's response to a particular treatment for multiple myeloma including, for example, the likelihood of the patient responding to the treatment, the likelihood of the patient relapsing after the treatment, and the durability of the patient's response to the treatment. In the case of immune modulating treatments, existing criteria for assessing patient response also fail to reflect the treatment's effect on the immune system. While some data suggests that recovery to normal immunoglobulin levels can be an indicator of some immune recovery, this metric is confounded with the use of maintenance therapies that impact immunoglobulin levels such as anti CD38 antibodies.

Disclosed are methods for determining immune stability for a subject based at least in part on dynamic time series data based on markers present in a subject's blood. In some example embodiments, a dynamical system model representative of an immune system may be determined. In some examples, the dynamical system representative of an immune system of a subject who is a patient having a cancer may be based on circulating cancer cells of a patient with multiple myeloma may be generated based on time series data obtained using a quantity of one or more antibodies present in a blood sample of the patient. For example, cytometry by time of flight (CyTOF) may be performed on the blood sample of the patient to determine the quantity of the markers present in the sample which are reactive to one or more antibodies. Moreover, the quantity of the one or more markers present in the blood sample which are responsive to antibodies may include a percentage of the one or more antibodies in the peripheral blood mononuclear cells (PBMCs) present in the blood sample of the patient. Examples of the one or more antibodies may include ANTI-FITC (CD38 multi-epitope), CD86/B7.2, CD45, CD80/B7-1, CD127/IL.7Ra, CD27, CD3, CD19, CD69, CD138/Syndecan 1, CD25, CD64, CD33, GRANZYME B, CD15/SSEA-1, CD8, HLA-DR, CD45RA, CD279/PD-1, CD14, CD56, CD223/LAG-3, TIGIT, CD269 (BCMA), CD319 (CS-1), gammadelta2 (TCR Vdelta2), CD163, TIM-3, CD16, CD4, CD274/PDL1, CD197/CCR7, CD11b, IgG-Kappa, IgG-Lambda, M-protein and CD20.

In some implementations, various other methods of determining a dynamic change in marker levels in the blood may be used for generating the dynamical model representative of the immune system of the subject. For example, in some implementations, immune populations may be determined based on complete blood count (CBC) data, differential white blood cell count data, Spectral Flow Cytometry (Cytek), and/or Mass Cytometry (CyTOF).

For example, in some implementations, CBC data which can be widely available, inexpensively obtained, and provide a less complex assay for blood analysis can be used to provide longitudinal total counts of red blood cells (RBC), white blood cells (WBC), and platelets using automated impedance or optical systems. The CBC data can also be used for determining immune stability of a subject.

In some implementations, a dynamical immune system model can be constructed based on differential white blood cell count data which expands upon CBC data by categorizing white blood cells (WBCs) into five major classes: neutrophils, lymphocytes, monocytes, eosinophils, and basophils. Performed by automated hematology based on cell size, granularity, and staining, this assay is low-cost and common in clinical practice, while providing good accuracy for major cell types but limited resolution for rare specific immune populations.

In some implementations, the time series data used for generating the dynamical immune system model can be based at least in part on spectral flow cytometry (Cytek) data. Spectral flow cytometry is an antibody-based assay that uses fluorophore-labeled antibodies to identify multiple immune cell subsets simultaneously. Instruments such as Cytek Aurora employ full-spectrum detection and computational unmixing to distinguish overlapping signals, enabling panels of 20-40 markers, which can be multiplexed to measure many combinations. This method is moderately expensive and primarily used in research and specialized clinical labs. Accuracy is high for well-designed panels, though compensation and spectral complexity require expertise.

In some implementations, the time series data for generating the dynamical immune system model can be based at least in part on mass cytometry (CyTOF). CyTOF represents the most complex and costly approach, leveraging antibodies conjugated to heavy metal isotopes and time-of-flight mass spectrometry for single-cell analysis. This technology enables simultaneous measurement of 40+ markers without spectral overlap, providing high resolution of immune populations, which may additionally be multiplexed. CyTOF is rarely used in routine clinical settings due to its high cost and technical demands, but it offers exceptional accuracy and dimensionality for research applications.

In some cases, in addition to and/or instead of the time series data of the quantity of one or more markers responsive to antibodies present in the blood sample of the patient, a dynamical system model representative of the immune system of the patient with a disease such as multiple myeloma may be generated based on time series data of a level of minimal residual disease present in the patient. The level of minimal residual disease (MRD) may be detected using a variety of techniques including, for example, multiparametric flow cytometry (MFC), allele-specific oligonucleotide (ASO)-qPCT, and next generation sequencing of VDJ sequences. As used herein, the term “minimal residual disease” refers to a quantity of tumor cells below the limit of detection available with conventional morphological assessment. In some instances, the term “minimal residual disease” may refer to (i) the quantity of tumor cells detected in the bone marrow using flow cytometry and/or next generation sequencing or (ii) the quantity of tumor cells detected outside of the bone marrow using sensitive imaging techniques. In the context of assessing a patient's response to a particular multiple myeloma treatment, “minimal residual disease negativity” is a response category in which (i) no tumor cells are detected in the bone marrow using flow cytometry and/or next generation sequencing or (ii) no tumor cells are detected outside of the bone marrow using sensitive imaging techniques.

In some example embodiments, the dynamical system model may include one or more functions, such as non-linear functions, corresponding to a derivative of the time series data of the quantity of the one or more markers (and/or the level of minimal residual disease) present in the blood sample of the patient. The derivative of the time series data may be measured or numerically approximated. In some cases, a regression may be performed to identify, within a library of candidate functions, the one or more functions for inclusion in the dynamical system model. In some implementations, a sparse regression may be used. For example, the library of candidate functions may include one or more constant functions, polynomial functions, and trigonometric functions of the measured data. Sparse regression may be performed to determine a sparse vector of coefficients indicating which one of the candidate functions included in the library of candidate functions is active. In some example embodiments, techniques including principal components analysis and/or the Nyquist criterion can be performed to identify, within a library of candidate functions, the one or more functions for inclusion in the dynamical model.

In some example embodiments, the steady state solution of the one or more functions included in the dynamical system model includes one or more eigenvalues. As such, the stability of the patient's immune system may be determined based on the one or more eigenvalues corresponding to the steady state solution of the one or more functions included in the dynamical system model. For example, the immune system of the patient and/or circulating cancer cells may be determined to be stable if the one or more eigenvalues do not have a threshold value (e.g., zero or a different threshold value). Alternatively, the immune system of the patient may be determined to be unstable if the one or more eigenvalues exceed the threshold value (e.g., zero or a different threshold value). Moreover, while the site of bone marrow biopsy doesn't show the presence of myeloma cells, circulating myeloma cells may come from different locations and represent better the general disease status. In some cases, the stability of the patient's immune system with or without the presence of circulating plasma cells may correspond to the short-term dynamics (e.g., dynamics occurring within a period of time such as an n-quantity of days, weeks, months, or years) of the patient's immune system. Alternatively, and/or additionally, the stability of the patient's immune system may correspond to the long-term dynamics (e.g., dynamics occurring beyond the period of time) of the patient's immune system.

In some example embodiments, the stability of the patient's immune system and/or circulating cancer cells may be used as a predictor of the patient's response to a treatment for multiple myeloma (e.g., an immune modulating treatment such as Daratumumab, CAR-T cells and/or the like). For example, the stability of the patient's immune system (e.g., stable or unstable) may be used to determine one or more of a first likelihood of the patient responding to the treatment, a second likelihood of the patient relapsing after the treatment, and a durability of the patient's response to the treatment. In some cases, a treatment plan for the patient may be determined based on the stability of the patient's immune system combined with the level of circulating cancer cells. For instance, the stability of the patient's immune system can indicate which immune based treatment is suitable for the patient and/or indicate the dosage and the timing of treatment. In some cases, the stability of the patient's immune system can further inform the treatment of the patient by indicating the length of the patient's response to a particular treatment including, for example, by categorizing the patient as a short-term responder or a long-term responder.

The dynamical system model representative of the immune system of the patient with multiple myeloma can be integrated with the quantity of circulating myeloma plasma cells present in the patient. The integration between changes in immune stability and residual circulating myeloma plasma cells present in patients could serve as easy read out for minimal residual disease (MRD) when patients are still considered in complete remission but need to be evaluate for depth of response and/or early progression A meta-analysis has confirmed that the depth of response can correlate with durability of response in MM. Specifically minimal residual disease can detect up to 10-6 levels or up to 10-6 MM cells in the marrow, patients who are MRD negative have longer progression free and overall survival. However, inherent immune function also plays a role in the ability of a patient to achieve and maintain a MRD negative state. Accordingly, a combination of circulating disease cells and immune system stability can be used in combination to establish the depth of response and easily monitoring disease the response over the time of treatment.

A number of clinical investigations have employed longitudinal sampling of peripheral blood mononuclear cells (PBMCs) to characterize systemic immune responses during treatment with immune checkpoint inhibitors such as anti-PD-1 or anti-PD-L1 antibodies. These studies typically use standardized workflows involving venipuncture, Ficoll-based PBMC isolation, cryopreservation, and later batch-processing through high-dimensional platforms such as mass cytometry (CyTOF), multicolor flow cytometry, and multiplex cytokine assays. Antibody panels in these studies commonly include lineage markers (e.g., CD3, CD4, CD8, CD19, CD14, CD56), differentiation or memory markers (e.g., CCR7, CD45RA/RO, CD27), myeloid markers (e.g., CD33, CD11b, HLA-DR), and inhibitory or activation markers (e.g., PD-1, PD-L1, TIM-3, CTLA-4, Ki-67, ICOS, CD38). Together these studies demonstrate the existence of established approaches for collecting, processing, and immunophenotyping PBMCs during immunotherapy.

Padrón et al. (2022) conducted a phase 1b/2 study in metastatic pancreatic ductal adenocarcinoma evaluating combinations of gemcitabine/nab-paclitaxel with nivolumab (anti-PD-1), sotigalimab (agonistic anti-CD40), or both agents. Longitudinal PBMC samples were collected at predefined time points, isolated by Ficoll, cryopreserved, and batch-analyzed. Immune profiling employed CyTOF mass cytometry and high-parameter fluorescence flow cytometry on a BD Symphony A5. The CyTOF and flow panels encompassed major T-cell subsets (CD4, CD8, memory and activation markers, PD-1, CTLA-4, Ki-67), B cells (CD19, CD27, regulatory B-cell markers), NK cells (CD56, CD16), monocytes and dendritic cells (CD14, CD11b, CD33, CD11c, HLA-DR), and additional markers of antigen presentation and checkpoint pathways. Serum cytokine and proteomic profiling (Olink) supplemented cellular readouts. The study demonstrates established methods for multi-platform, high-dimensional PBMC immunophenotyping during chemo-immunotherapy combinations. (Padrón, L. J., Maurer, D. M., O'Hara, M. H. et al. Sotigalimab and/or nivolumab with chemotherapy in first-line metastatic pancreatic cancer: clinical and immunologic analyses from the randomized phase 2 PRINCE trial. Nat Med 28, 1167-1177 (2022). https://doi.org/10.1038/s41591-022-01829-9)

Leung et al. (2021) performed a prospective longitudinal analysis of PBMCs from non-small cell lung cancer patients treated with anti-PD-1 monotherapy. PBMCs were collected from patients and healthy donors at baseline and multiple on-treatment time points, then immunophenotyped using a CyTOF panel containing approximately 37 antibodies. This panel included lineage markers (CD3, CD4, CD8, CD14, CD19, CD33, CD56), memory markers (CCR7, CD45RA/RO), B-cell and NK-cell markers, and functional proteins including PD-1, TIM-3, CD69, ICOS, CD101, Ki-67, and granzyme-related markers. Serum samples were also collected and assessed using MSD multiplex cytokine assays. PBMCs underwent standard Ficoll isolation, cryostorage, and batch CyTOF acquisition. Computational analysis included FlowSOM clustering and UMAP/t-SNE dimensionality reduction. This study exemplifies established PBMC-based immune monitoring in checkpoint inhibitor therapy using high-parameter CyTOF panels. (Leung, E. L H., Li, R Z., Fan, X X. et al. Longitudinal high-dimensional analysis identifies immune features associating with response to anti-PD-1 immunotherapy. Nat Commun 14, 5115 (2023). https://doi.org/10.1038/s41467-023-40631-0)

Huang et al. (2024) performed immune profiling within two large randomized clinical trials in locoregionally advanced nasopharyngeal carcinoma receiving chemoradiotherapy with or without anti-PD-1 therapy. PBMCs were collected longitudinally at multiple treatment stages and analyzed with mass cytometry using a comprehensive ˜37-marker CyTOF panel. The antibody panel included canonical markers for T-cell subsets (CD3, CD4, CD8, CD45RA/RO, CCR7, CD25, CD127), regulatory T cells (FOXP3, CD39), B cells (CD19, CD21, CD24), and myeloid populations (CD14, CD33, CD11c, CD16, HLA-DR). Additional functional markers for activation or checkpoint pathways (CD38, PD-1, PD-L1, TIM-3, CD86, CD40, Ki-67) were also included. Serum cytokines were assessed using MSD platforms and multiplex bead-based assays. Sample handling followed standardized PBMC processing, cryopreservation, and batch cytometry acquisition. This represents a large-scale, structured implementation of longitudinal CyTOF profiling for checkpoint inhibitor therapy in a randomized clinical setting. (Huang, S W., Jiang, W., Xu, S. et al. Systemic longitudinal immune profiling identifies proliferating Treg cells as predictors of immunotherapy benefit: biomarker analysis from the phase 3 CONTINUUM and DIPPER trials. Sig Transduct Target Ther 9, 285 (2024). https://doi.org/10.1038/s41392-024-01988-w)

van den Ende et al. (2023) conducted a longitudinal PBMC immunophenotyping study in patients with resectable esophageal adenocarcinoma treated with neoadjuvant chemoradiotherapy combined with anti-PD-L1 therapy. PBMC sampling occurred at baseline, on-treatment, pre-surgery, and post-surgery. Unlike the CyTOF-based studies, PERFECT utilized multicolor flow cytometry with 14-color panels supplemented by macrophage-focused secondary panels. These panels included markers for T-cell subsets (CD3, CD4, CD8, CD45RA, CD27, FOXP3, CTLA-4, PD-1, TIM-3, Ki-67), B cells (CD19, CD40, CD80/86), NK cells (CD56, CD16), monocyte subsets (CD14, CD16), dendritic cells (BDCA-2, BDCA-3, CD1c), and MDSC-related markers (CD33, CD11b, HLA-DR). Serum cytokines (e.g., TGF-β, IL-6, IL-8, CXCL9/10, CCL2/5, VEGF) were quantified by ELISA and bead-based assays. PBMCs were isolated by Ficoll gradient separation, cryopreserved, and analyzed in batch to minimize technical variability. This study illustrates the use of standardized multicolor flow cytometry panels for tracking systemic immune profiles during neoadjuvant immunotherapy. (van den Ende T, Ezdoglian A, Baas L M, Bakker J, Lougheed S M, Harrasser M, Waasdorp C, van Berge Henegouwen M I, Hulshof M C C M, Haj Mohammad N, van Hillegersberg R, Mook S, van der Laken C J, van Grieken N C T, Derks S, Bijlsma M F, van Laarhoven H W M, de Gruijl T D. Longitudinal immune monitoring of patients with resectable esophageal adenocarcinoma treated with Neoadjuvant PD-L1 checkpoint inhibition. Oncoimmunology. 2023 Jul. 17; 12 (1): 2233403. doi: 10.1080/2162402X.2023.2233403. PMID: 37470057; PMCID: PMC10353329.)

The contents of Padron et al. 2022, Leung et al. (2021), Huang et al. (2024), and van den Ende et al. (2023), are hereby incorporated by reference in their entirety.

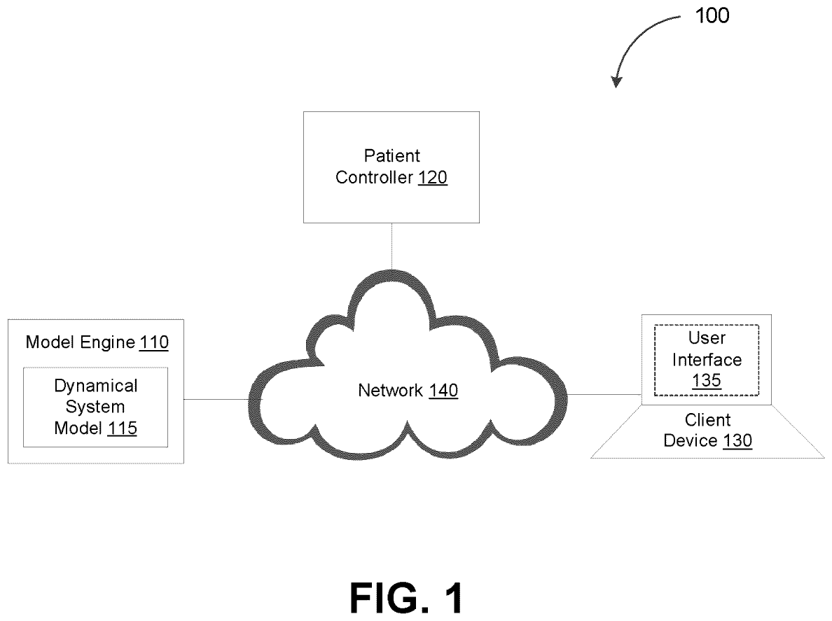

FIG. 1 depicts a system diagram illustrating an example of an analysis system 100, in accordance with some example embodiments. Referring to FIG. 1, the analysis system 100 may include a model engine 110, a patient controller 120, and a client device 130. As shown in FIG. 1, the model engine 110, the patient controller 120, and the client device 130 may be communicatively coupled via a network 140. The client device 130 may be a processor-based device including, for example, a workstation, a desktop computer, a laptop computer, a smartphone, a tablet computer, a wearable apparatus, and/or the like. The network 140 may be a wired network and/or a wireless network including, for example, a local area network (LAN), a virtual local area network (VLAN), a wide area network (WAN), a public land mobile network (PLMN), the Internet, and/or the like.

In some example embodiments, the model engine 110 may generate, based on time series data, a dynamical system model 115 representative of an immune system of a subject. In some cases, the time series data may be of a quantity of one or more antibodies or circulating cancer cells present in a blood sample of a patient with multiple myeloma. Alternatively, and/or additionally, the time series data may be of a level of minimal residual disease (MRD) present in the patient. The level of minimal residual disease may correspond to, for example, a quantity of tumor cells present in the bone marrow of the patient as detected using flow cytometry and/or next generation sequencing. In some cases, the level of minimal residual disease may also correspond to a quantity of tumor cells present outside of the bone marrow of the patient in combination with specific immune subsets as detected using sensitive imaging techniques. Alternatively and/or additionally, the time series data may be of levels of biomarkers for immune cells in a healthy subject.

In instances where the time series data is of the quantity of one or more antibodies present in the blood sample of the patient with multiple myeloma, the time series data may correspond to a cytometry by time of flight (CyTOF) panel containing a customized selection of antibodies shown in Table 1 below. The cytometry by time of flight (CyTOF) panel may indicate, at each successive time points (e.g., successive days, weeks, months, and/or the like), a percentage of the one or more antibodies in a plurality of peripheral blood mononuclear cells (PBMCs) present in the blood sample of the patient. Accordingly, the model engine 110 may generate the dynamical system model 115 based the percentage of the one or more antibodies in a plurality of peripheral blood mononuclear cells (PBMCs) present in the blood sample of the patient at a sequence of successive time points. The resulting dynamical system model 115 may include one or more functions characterizing the state of the patient's immune system.

TABLE 1
Multiple Myeloma CyTOF Panel

| Ab | Clone |
|---|---|
| ANTI-FITC (CD38 Multi-epitope) | FIT-22 |
| CD86/B7.2 | IT2.2 |
| CD45 | HI30 |
| CD80/B7-1 | 2D10.4 |
| CD127/IL.7Ra | A019D5 |
| CD27 | L128 |
| CD3 | UCHT1 |
| CD19 | HIB19 |
| CD69 | FN50 |
| CD138/Syndecan 1 | DL-101 |
| CD25 | 2A3 |
| CD64 | 10.1 |
| CD33 | WM53 |
| GRANZYME B | GB11 |
| CD15/SSEA-1 | W6D3 |
| CD8 | SK1 |
| HLA-DR | L243 |
| CD45RA | HI100 |
| CD279/PD-1 | EH12.2H7 |
| CD14 | M5E2 |
| CD56 | NCAM 16.2 |
| CD223/LAG-3 | 11C3C65 |
| TIGIT | MBSA43 |
| CD269 (BCMA) | 19F2 |
| CD319 (CS-1) | CRACC |
| gammadelta2 (TCR Vdelta2) | B6 |
| CD163 | GHI/61 |
| TIM-3 | F38-2E2 |
| CD16 | 3G8 |
| CD4 | SK3 |
| CD274/PDL1 | 29E.2A3 |
| CD197/CCR7 | G043H7 |
| CD11b | ICRF44 |
| IgG-Kappa | Rabbit PAb (agilent #A0191) |
| IgG-Lambda | Rabbit PAb (agilent #A0193) |
| CD20 | 2H7 |

As discussed above, in some example embodiments, the model engine 110 may generate, based on time series data corresponding to a cytometry by time of flight (CyTOF) panel, a dynamical system model 115 representative of an immune system of the patient. However, cytometry by time of flight (CyTOF) panels and the like can produce vast amounts of data, corresponding, for example, to over fifty, seventy, or hundred populations of cells, which may make it difficult to select a model that is explanatory. Accordingly, in some embodiments, the time series data can be pre-processed before the model engine 110 generates the dynamical system model 115. For example, data dimensionality reduction techniques such as principal components analysis (PCA) can be used. In some example embodiments PCA can be used to identify subpopulations of cells which contribute to the unique behavior of the individual populations.

Using PCA, the original matrix with cell types and observations can be decomposed into three matrices. This can be represented as follows: A=UΣV^{T}, where A is the original matrix with columns of cell types and rows of individual observations, U is the left singular vectors, Σ is a diagonal matrix containing the singular values, and V^{T} is the right singular vectors. The values on the diagonal of Σ indicate the amount of the total variation in the dataset which the decomposition accounts for, and the rows in V^{T} describe the amount that each cell type contributes to each component of the decomposition. The decomposition of the cell type and observation data into these decomposed matrices can allow for the identification of the principal components or cell types that contribute most to the variation in the dataset. For example, in some embodiments, the first four identified principal components or cell types may account for over eighty percent of the total variation in the database. Accordingly, the identified principal components can be provided to the model engine 110 rather than the larger cell type dataset. In this manner, a subset of cell types can be identified for filtering the CyTOF panel for further analysis by the model engine 110. This process may provide computational efficiencies.

In some example embodiments, the model engine 110 may generate the dynamical system model 115 representative of the immune system of the patient by at least identifying the one or more functions for inclusion in the dynamical system model 115. For example, the one or more functions selected for inclusion in the dynamical system model 115 may correspond to a derivative of the time series data, which may be measured or approximated based on the times series data. Moreover, the one or more functions may be non-linear functions representative of the non-linear dynamics exhibited by the immune system of the patient. These non-linear functions may be selected from a library of candidate functions that includes one or more constant functions, polynomial functions, and trigonometric functions. In some cases, the model engine 110 may identify the one or more functions for inclusion in the dynamical system model 115 by at least performing a sparse regression to determine a sparse vector of coefficients indicating which functions in the library of candidate functions are active. That is, the model engine 110 may perform the sparse regression to achieve a sparse solution that minimizes the quantity of functions included in the dynamical system model 115 representative of the immune system. Accordingly, the resulting dynamical system model 115 may balance model complexity, as measured in the quantity of functions included in the dynamical system model 115, and model accuracy, as measured by how well the dynamical system model 115 is able to predict the behavior of the patient's immune system.

To further illustrate, the dynamical system model 115 may be expressed in terms of Equation (1) below, in which the vector x(t)∈ [symbol] denotes the state of the patient's immune system at time t, and the f(x(t)) denotes the governing functions selected for inclusion in the dynamical system model 115. The functions f(x(t)) may therefore represents the dynamic constraints that define the changes in the state of the patient's immune system.

$$\frac{d}{dt} x ( t ) = f ( x ( t ) ) \qquad ( 1 )$$

The model engine 110 may identify the functions f based on time series data that includes the state x(t) of the patient's immune system at successive time points. For example, the state x(t) of the patient's immune system at each time point may include a quantity of the one or more antibodies, such as those included in the aforementioned cytometry by time of flight (CyTOF) panel, present in a blood sample of the patient at that time point. Alternatively and/or additionally, the state x(t) of the patient's immune system at each time point may include a level of minimal residual disease (MRD), as indicated by the quantity of tumor cells present in and/or outside of the bone marrow of the patient. The model engine 101 may identify the functions fbased on a derivative \dot{x}(t) of the time series data. For instance, the model engine 110 may either measure the derivative \dot{x}(t) of the time series data directly or approximately the derivative \dot{x}(t) of the time series data from successive states x(t) of the patient's immune system. Examples of the time series data X including n states x(t) of the patient's immune system at m successive time points and the corresponding derivative X are shown as matrices (2) and (3) below.

$$X = [ \begin{matrix}{x}^{T} ( {t}_{1} ) \\ {x}^{T} ( {t}_{2} ) \\ ⋮ \\ {x}^{T} ( {t}_{m} )\end{matrix} ] = [ \begin{matrix}{x}_{1} ( {t}_{1} ) & {x}_{2} ( {t}_{1} ) & … & {x}_{n} ( {t}_{1} ) \\ {x}_{1} ( {t}_{2} ) & {x}_{2} ( {t}_{2} ) & … & {x}_{n} ( {t}_{2} ) \\ ⋮ & ⋮ & ⋱ & ⋮ \\ {x}_{1} ( {t}_{m} ) & {x}_{2} ( {t}_{m} ) & … & {x}_{n} ( {t}_{m} )\end{matrix} ] \qquad ( 2 )$$

$$\dot{X} = [ \begin{matrix}{\dot{x}}^{T} ( {t}_{1} ) \\ {\dot{x}}^{T} ( {t}_{2} ) \\ ⋮ \\ {\dot{x}}^{T} ( {t}_{m} )\end{matrix} ] = [ \begin{matrix}{\dot{x}}_{1} ( {t}_{1} ) & {\dot{x}}_{2} ( {t}_{1} ) & … & {\dot{x}}_{n} ( {t}_{1} ) \\ {\dot{x}}_{1} ( {t}_{2} ) & {\dot{x}}_{2} ( {t}_{2} ) & … & {\dot{x}}_{n} ( {t}_{2} ) \\ ⋮ & ⋮ & ⋱ & ⋮ \\ {\dot{x}}_{1} ( {t}_{m} ) & {\dot{x}}_{2} ( {t}_{m} ) & … & {\dot{x}}_{n} ( {t}_{m} )\end{matrix} ] \qquad ( 3 )$$

The model engine 110 may identify the functions f from a library Θ(X) of candidate nonlinear functions, with each column in the library Θ(X) may corresponding to a candidate function. As shown in matrix (4) below, the library Θ(X) may contain a selection of constant functions, polynomial functions, and trigonometric functions. It should be appreciated that higher order polynomials in the library Θ(X) are denoted as X^{P}^{₂}, X^{P}^{₃}, and/or the like. For example, the polynomial X^{P}^{₂} may denote the quadratic nonlinearities in present in the state x of the patient's immune system. In some cases, the library Θ(X) may include a p quantity of candidate functions, which is fewer than the m time points for which included in the time series data X (e.g., p<<m).

$$Θ ( X ) = [ \begin{matrix}│ & │ & │ & │ & & │ & │ & \\ 1 & X & {X}^{{P}_{2}} & {X}^{{P}_{3}} & … & sin X & cos X & … \\ │ & │ & │ & │ & & │ & │ & \end{matrix} ] \qquad ( 4 )$$

The model engine 110 may perform sparse regression in order to identify a few active functions in the library Θ(X) as the functions f included in the dynamical system model 115. For example, the model engine 110 may apply perform sparse regression to determine sparse vectors of coefficients Ξ=[ξ₁ ξ₂ . . . ξₙ], which determines which candidate functions in the library Θ(X) are active. That is, as indicated by Equation (5) below, the derivative \dot{X} may be modeled by the candidate equations in the library Θ(X) identified as active by the sparse vectors of coefficients E.

$$\dot{X} = Θ ( X ) Ξ \qquad ( 5 )$$

In some example embodiments, in addition to the quantity of one or more antibodies present in the blood sample of the patient (e.g., the customized selection of antibodies forming the cytometry by time of flight (CyTOF) panel shown in Table 1), the model engine 110 may identify the one or more functions ffor inclusion in the dynamical system model 115 of the immune system of the patient based on the quantity of circulating myeloma plasma cells present in the blood sample of the patient. In some cases, the quantity of circulating myeloma plasma cells present in the blood sample of the patient may be indicative of the patient's level of minimal residual disease (MRD). Furthermore, in some instances, the level of minimal residual disease for the patient may be determined based on a stability of the patient's immune system. As will be described in more detail below, the stability of the patient's immune system may be determined based on the dynamical system model 115 of the patient's immune system.

In some example embodiments, the model engine 110 may identify the one or more functions f for inclusion in the dynamical system model 115 using techniques including principal components analysis and/or the Nyquist criterion.

In some example embodiments, generating the dynamical system model includes filtering the library of candidate functions to exclude functions within the library of candidate functions having an oscillation frequency exceeding two times the maximum sampling frequency of the data.

The model engine 110 may apply a Nyquist Sampling Criterion to prevent inclusion of active functions in the library Θ(X) that may be prone to overfitting from the functions f included in the dynamical system model 115. In some embodiments, the library Θ(X) may include rapid oscillatory behaviors that are prone towards overfitting the data. The Nyquist Sampling Criterion states that the highest frequency measured in a signal must be measured at twice the highest frequency to prevent aliasing. Conversely, frequencies which are higher than twice the greatest sampling frequency are not supported by the data, regardless of how well it fits the data. Thus, the Nyquist Sampling Criterion can be applied to filter the library Θ(X) and prevent functions that overfit from being included in the functions f included in the dynamical system model 115.

To remove models which contain oscillatory frequencies higher than twice the greatest sampling frequency, the model engine 110 may obtain the fastest sampling frequency from the data. Assuming multidimensional timeseries data x, and a matrix of model coefficients A, the eigenvalues of the model coefficients are given as follows λ=α±jβ, where alpha is the real component of the eigenvalue, and beta is the imaginary component. Eigenvalues with purely real components are non-oscillatory, but eigenvalues with |β|>0 contribute oscillatory behavior to the model. The frequency of the oscillations from the model are:

$$f = \frac{\vert β \vert}{2 π} .$$

As such, any models for which the frequency of the oscillations f exceeds twice the maximum sampling frequency of the data can be filtered from the library of functions and excluded from the functions f that are included in the dynamical system model 115. Specifically, if any (|β|)≥πƒ_{max,sample}→remove from potential models.

In some example embodiments, once the dynamical system model 115 is generated to include the functions f, the patient controller 120 may apply the dynamical system model 115 to determine the stability of the patient's immune system. In some cases, the stability of the patient's immune system may be determined based on a stable state solution of the functions f included in the dynamical system model 115. For example, the patient controller 120 may perform a linear stability analysis to determine the linear stability of the functions f. The linear approximation of the steady state solution to the functions f has the form

$$\frac{dr}{dt} = Ar ,$$

wherein r denotes the perturbation to the steady state of the system and A denotes a linear operator. Where the spectrum of the linear operator A contains eigenvalues with a positive real portion, the immune system of the patient as characterized by the functions f is linearly unstable. Contrastingly, if the spectrum of the linear operator A contains eigenvalues with a negative real portion, the immune system of the patient as characterized by the functions f is linearly stable. That is, the patient controller 120 may determine that the dynamics of the patient's immune system is unstable if the eigenvalues forming the steady state solution of the governing functions f are greater than zero and stable if the eigenvalues are less than or equal to zero.

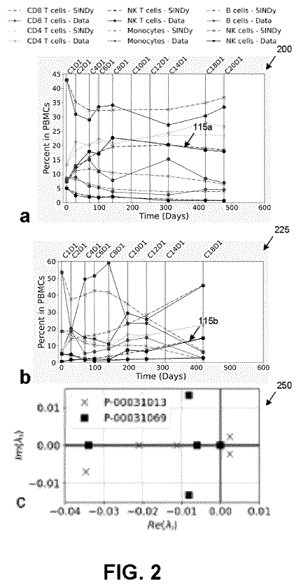

FIG. 2 depicts graphs illustrating an immune stability analysis for two example patients, in accordance with some example embodiments. For example, FIG. 2(a) depicts a graph 200 illustrating the state of Patient P-00031069's immune system as indicated by the quantity of one or more antibodies, such as the customized selection of antibodies forming the cytometry by time of flight (CyTOF) panel shown in Table 1, in the peripheral blood mononuclear cells (PBMCs) present in the blood sample of Patient P-00031069. Each dotted line in the graph 200 plots the changes the quantity of one antibody in the peripheral blood mononuclear cells (PBMCs) present in the blood sample of Patient P-00031069. Each solid line in the graph 200 plots the changes in the quantity of the same antibody as determined by the corresponding governing function f included in a first dynamical system model 115a associated with Patient P-00031069.

FIG. 2(b) depicts graph 225 in which each dotted line plots the changes in the quantity of one particular antibody in the peripheral blood mononuclear cells (PBMCs) present in the blood sample of Patient P-00031013. Meanwhile, each solid line in the graph 225 plots the changes in the quantity of the same antibody as determined by the corresponding governing function f included in a second dynamical system model 115b associated with Patient P-00031013.

FIG. 2(c) depicts a graph 250 plotting the real Re(λᵢ) and imaginary Im(λᵢ) portions of the eigenvalues forming the steady state solutions of the governing functions f governing functions f included in the first dynamical system model 115a associated with Patient P-00031069 and the second dynamical system model 115b associated with Patient P-00031013. As shown in FIG. 2(c), the short- and long-term dynamics of the immune system of Patient P-00031069 may be determined to be stable based on the eigenvalues forming the steady state solution of the first dynamical system model 115a of Patient P-00031069 being equal to or less than zero (e.g., Re(λᵢ)≤0). By contrast, FIG. 2(c) shows that the short- and long-term dynamics of the immune system of Patient P-00031013 may be determined to be unstable based on the eigenvalues forming the steady state solution of the second dynamical system model 115b of Patient P-00031013 being greater than zero (e.g., Re(λᵢ)>0).

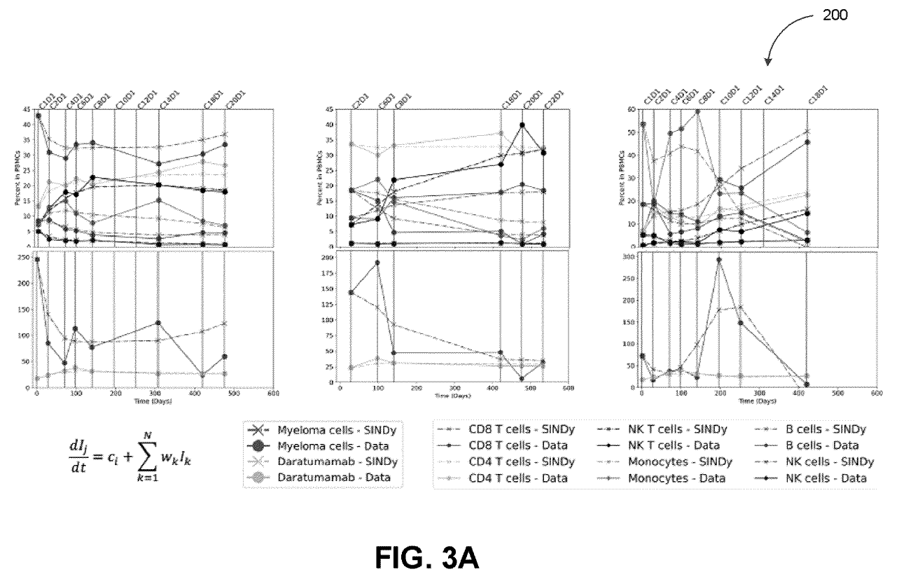

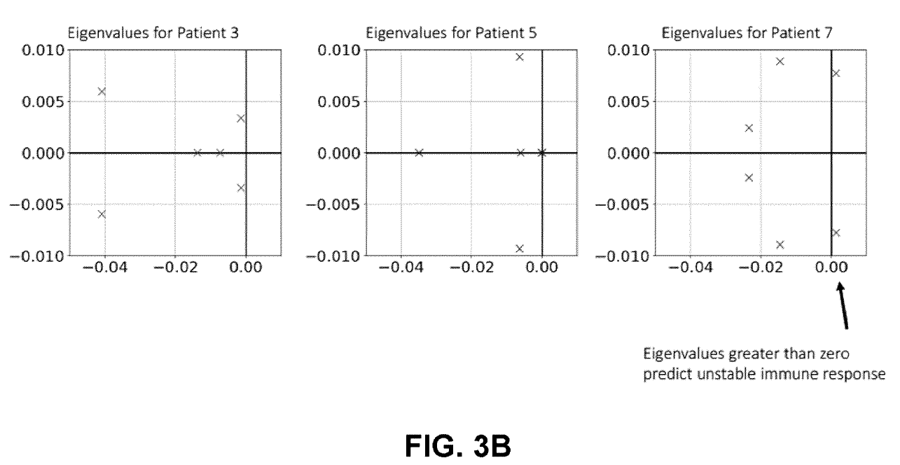

FIGS. 3A-B depict an immune stability analysis for three additional example patients. For example, FIG. 3A depicts graphs in which the dotted lines correspond to the quantities of various antibodies present in the blood samples of each patient and the quantities of the same antibodies as determined by the corresponding governing functions f are plotted in solid lines. FIG. 3B plots the eigenvalues forming the steady state solutions of the governing functions f.

In some example embodiments, the patient controller 120 may apply the dynamical system model 115 to assess, based on the stability of the patient's immune system, the patient's response to one or more treatments for multiple myeloma. It should be appreciated that immune stability analysis and possibly circulating cancer cells may be performed based on the patient's blood samples taken before, during and/or after the patient has undergone a first treatment for multiple myeloma, such as an immune modulating drug (e.g., Daratumumab, CAR T-cells, and/or the like). Accordingly, before, during, and/or after the patient has undergone the first treatment for multiple myeloma, the patient controller 120 may apply the dynamical system model 115 to determine, based on a stability of the patient's immune system, a first likelihood of the patient responding to the first treatment, a second likelihood of the patient relapsing after the first treatment, and a durability of the patient's response to the first treatment. Where the patient is determined as unlikely to respond to the first treatment or likely to relapse after the first treatment, or in instances where the patient's response to the first treatment is determined to lack durability, the patient controller 120 may identify a second treatment for the patient. In some instances, in addition to identifying one or more multiple myeloma treatments for the patient, the patient controller 120 may further apply the dynamical system model 115 to determine, based on the stability of the patient's immune system, a timing and/or a dosage of the treatments. It should be appreciated that at least a portion of the analysis performed by the patient controller 120 based on the dynamical system model 115 may be displayed, for example, as a part of a user interface 135 at the client device 130.

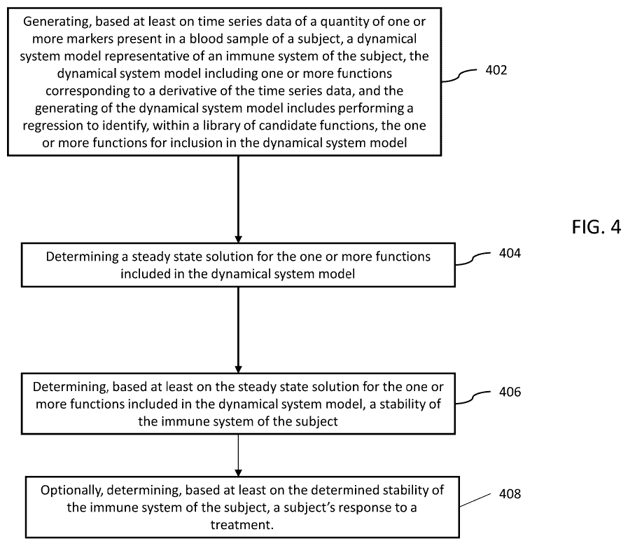

FIG. 4 depicts a flowchart illustrating an example of a process 400 for immune stability analysis, in accordance with some example embodiments. Referring to FIGS. 1 and 4, the process 400 may be performed by the model engine 110 and the patient controller 120. For example, the model engine 110 may generate the dynamical system model 115 representative of the immune system of a subject and the patient controller 120 may apply the dynamical system model 115 to determine a stability of the immune system of the subject.

At 402, a dynamical system model representative of an immune system of a subject (e.g., healthy subject, patient with cancer, patient with multiple myeloma) may be generated based at least on time series data of a quantity of one or more biomarkers responsive to antibodies present in a blood sample of the subject. In some example embodiments, the model engine 110 may generate the dynamical system model 115 by at least identifying, from the library Θ(X) of candidate nonlinear functions, one or more functions f representative of the dynamics of the subject's immune system. For example, the one or more functions f may be identified based on time series data that includes, for each time point in a sequence of successive time points, a state x(t) of the subject's immune system. In some cases, the state x(t) of the subject's immune system at any one time point may be characterized by the quantity of one or more markers responsive to antibodies, such as the customized selection of antibodies forming the cytometry by time of flight (CyTOF) panel shown in Table 1, in a plurality of peripheral blood mononuclear cells (PBMCs) present in a blood sample of the subject. In particular, the model engine 110 may identify one or more functions f based on a measured or approximated derivative x(t) of the time series data. For instance, the model engine 110 may perform a regression (e.g., a sparse regression) in order to determine the sparse vectors of coefficients E, which indicates the active functions in the library Θ(X) for inclusion in the dynamical system model 115. Accordingly, as shown in FIGS. 2 and 3A-B, the one or more functions f may model the changing states x(t) of the subject's immune system characterized, for example, by the changing quantities of one or more antibodies in the peripheral blood mononuclear cells (PBMCs) present in the blood sample of the patient.

At 404, a steady state solution for one or more functions included in the dynamical system model may be determined. In some example embodiments, the patient controller 120 may perform a linear stability analysis to determine the linear stability of the functions f. The linear approximation of the steady state solution to the functions f has the form

$$\frac{dr}{dt} = Ar ,$$

wherein r denotes the perturbation to the steady state of the system and A denotes a linear operator.

At 406, a stability of the immune system of the subject may be determined based at least on the steady state solution for the one or more functions included in the dynamical system model. In some example embodiments, the patient controller 120 may determine, based on a steady state solution for the functions f included in the dynamical system model 115 of the subject's immune system, a stability of the subject's immune system. For example, the steady state solution of the functions f included in the dynamical system model 115 of the subject's immune system may include eigenvalues having a real portion Re(λᵢ) and an imaginary portion Im(λᵢ). That is, the spectrum of the linear operator A forming the linear approximation of the steady state solution may contain eigenvalues having either a positive or a negative real portion Re(λᵢ). Where the real portion Re(λᵢ) of the eigenvalues forming the steady state solution of the functions f included in the dynamical system model 115 of the subject's immune system is greater than zero (e.g., Re(λᵢ)>0), the patient controller 120 may determine that the short- and long-term dynamics of the subject's immune system is unstable. Contrastingly, if the real portion Re(λᵢ) of the eigenvalues forming the steady state solution of the functions f included in the dynamical system model 115 of the subject's immune system is equal to or less than zero (e.g., Re(λᵢ)≤0), the patient controller 120 may determine that the short- and long-term dynamics of the subject's immune system is stable.

At 408, the subject's response to a treatment (e.g., a treatment for multiple myeloma) may be determined based on the stability of the subject's immune system. For instance, in some example embodiments, the patient controller 120 may determine, based at least on the stability of the subject's immune system, a first likelihood of the subject responding to the first treatment, a second likelihood of the subject relapsing after the first treatment, and a durability of the subject's response to the first treatment. In instances where the first likelihood of the subject responding to the first treatment, the second likelihood of the subject relapsing after the first treatment, and/or the durability of the subject's response to the first treatment fail to satisfy one or more thresholds, the patient controller 110 may identify a second treatment for the subject. Furthermore, in some cases, the patient controller 120 may further apply the dynamical system model 115 to determine, based on the stability of the subject's immune system, a timing and/or a dosage of the treatments.

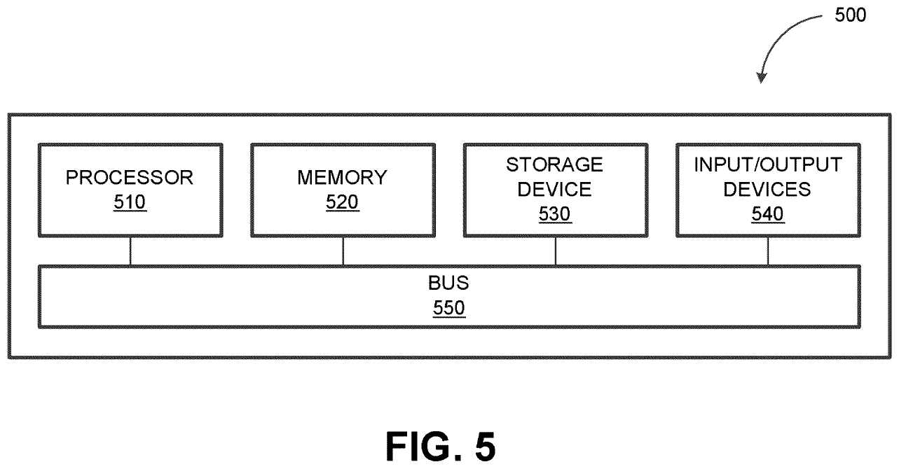

FIG. 5 depicts a block diagram illustrating an example of computing system 500, in accordance with some example embodiments. Referring to FIGS. 1 and 5, the computing system 500 may be used to implement the model engine 110, the patient controller 120, the client device 130, and/or any components therein.

As shown in FIG. 5, the computing system 500 can include a processor 510, a memory 520, a storage device 530, and input/output devices 540. The processor 510, the memory 520, the storage device 530, and the input/output devices 540 can be interconnected via a system bus 550. The processor 510 is capable of processing instructions for execution within the computing system 500. Such executed instructions can implement one or more components of, for example, the model engine 110, the patient controller 120, the client device 130, and/or the like. In some example embodiments, the processor 510 can be a single-threaded processor. Alternately, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, or a tape device, or other suitable persistent storage means. The input/output device 540 provides input/output operations for the computing system 500. In some example embodiments, the input/output device 540 includes a keyboard and/or pointing device. In various implementations, the input/output device 540 includes a display unit for displaying graphical user interfaces.

According to some example embodiments, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example embodiments, the computing system 500 can be used to execute various interactive computer software applications that can be used for organization, analysis and/or storage of data in various formats. Alternatively, the computing system 500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

### EXPERIMENTAL DATA

### Experiment #1

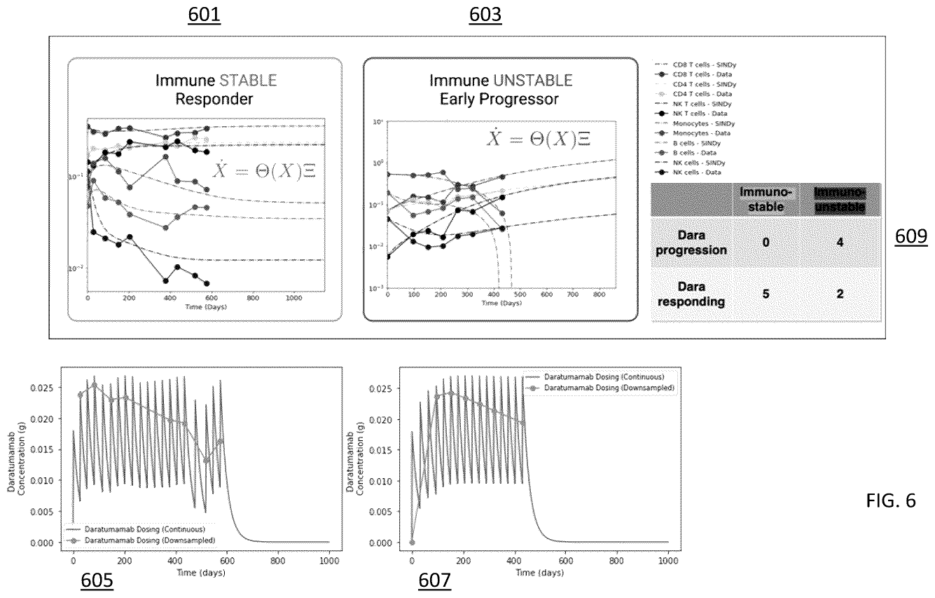

FIGS. 6-16 provide experimental data from application of one or more aspects or features of the subject matter described herein. For example, a system for immune stability and circulating plasma cell analysis was applied to retrospective longitudinal patient data from eleven patients. The eleven patients underwent an autologous stem cell transplant and were provided with daratumumab (sometimes referred to as “dara”) for maintenance. Cell population counts were identified using CyTOF at multiple time points corresponding to dara treatment dates. Of this population, four patients had progressive disease, while seven patients had stable disease over the course of the trial. The concentration of daratumumab is modeled using the half-life of daratumumab within the body. Using eigenvalue analysis on the major immune supertypes as well as the modeled concentration of daratumumab, the instability of the immune system dynamics was modeled by a system for immune stability and circulating plasma cell analysis built in accordance with the description herein. The system was able to accurately classify four of the four patients with progressive disease (100% sensitivity) and identified five of the seven patients with stable disease as having stable immune dynamics (71% specificity). With a prevalence of progression of four of the eleven patients, these results have a p-value of 0.0625 (given a null hypothesis that the sensitivity of the method to detecting disease progression is 50%).

FIG. 6 illustrates results from a system for immune stability and circulating plasma cell analysis applied to retrospective longitudinal patient data from eleven patients. As shown, there are differences between a strong dara-responder 601 (immune stable as identified by the platform described herein, stable disease clinically) among the six largest immune cell supertypes illustrated in the dataset when compared to an immune unstable early progressor 603. In FIG. 6 the cell dynamics are illustrated alongside the patient's individual time-resolved dara concentration within the drug. For example, dara concentrations for an immune stable responder 605 and dara concentrations for an immune unstable early progressor 607 are shown. Also displayed are initial results 609 from the disclosed systems, which indicate 100% sensitivity to detecting early progression, and 71% specificity in ruling out early progressing.

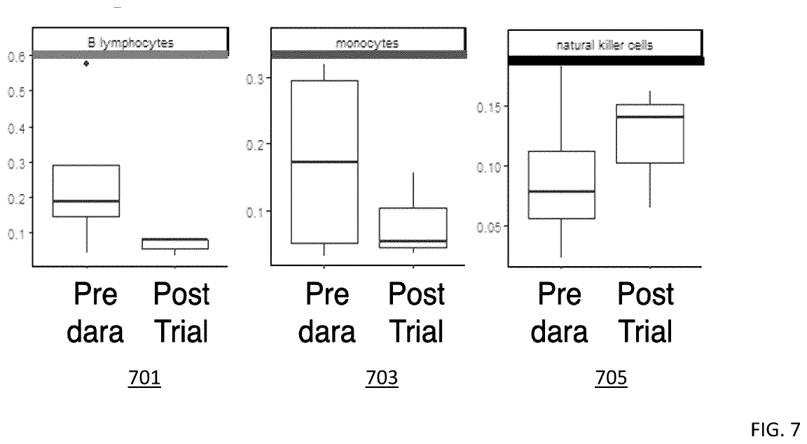

FIG. 7 illustrates validation of predictions made by a system for immune stability and circulating plasma cell analysis as applied to retrospective longitudinal patient data from the eleven patients discussed above. Predictions made by a system for immune stability and circulating plasma cell analysis for patients predicted to have progressive disease state may correspond to an extinction of lymphocytes and monocytes and an increase in natural killer T-cells. FIG. 7 validates the predictions output by the models described herein with single-cell RNA sequencing. Illustrated in FIG. 7 is single-cell RNA sequencing data, acquired prior to entering a trial using dara for maintenance, and immediately after exiting the trial. Shown are preliminary single-cell RNA sequencing data on three progressing patients pre- and post-dara maintenance. In patients whose disease progresses, cell populations of b-lymphocytes 701 and monocytes 703 are nearly extinct, while natural killer cells 705 increase in population. These results validate the progression of disease forecast by the models described herein.

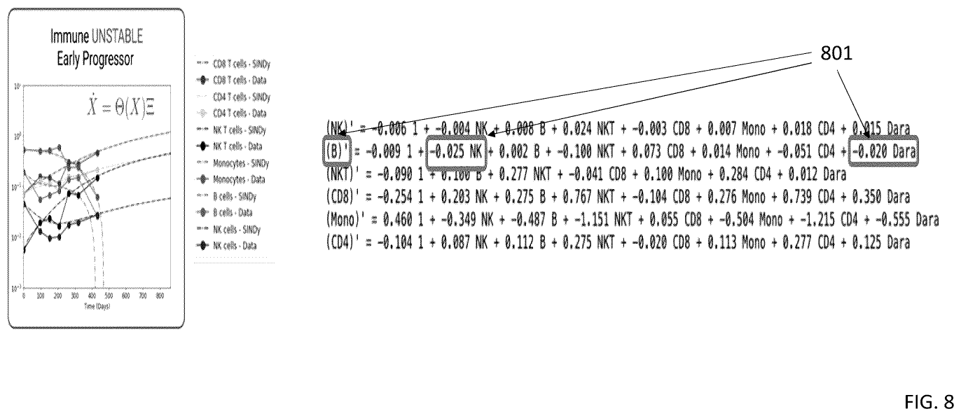

FIG. 8 illustrates that the disclosed systems and methods can be used for the discovery of novel immune cell interactions and off-target dara effects. As illustrated in FIG. 8, the dynamic model identified by the techniques described herein may predict B-lymphocyte and monocyte extinction and natural killer cell proliferation. The model can also identify interactions between off-target cell types 801. In the illustrated example, B-lymphocyte extinction may be partially due to dara toxicity, as well as other immune interactions.

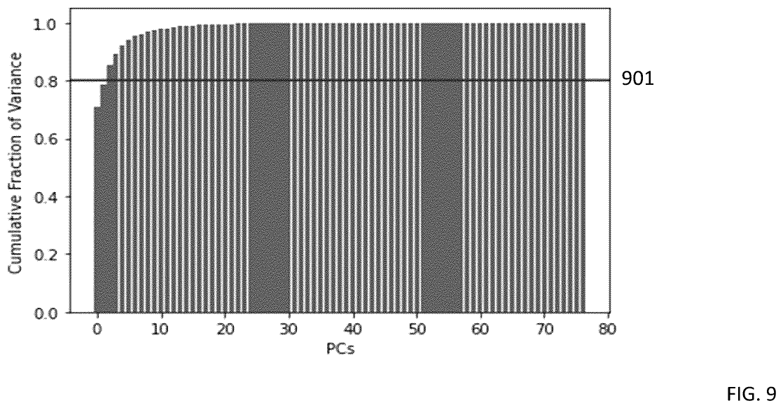

FIG. 9 illustrates an application of reducing the dimensionality of the time series data by applying principal components analysis. Principal components analysis was applied to the cyTOF data from the eleven patients that underwent an autologous stem cell transplant and were provided with dara for maintenance. Using principal components analysis, the first four principal components were shown to account for the majority of the variation. Accordingly, the disclosed systems were run using cell data corresponding to the first four principal components and found to produce similar results. Accordingly, principal components can be used to reduce dimensionality of the dataset for more efficient results while retaining the informational value.

FIG. 9 shows the cumulative fraction of variance over the principal components. As shown, a line 901 is drawn at 0.8 indicating that only three principal components account for more than 80% of the total variation in the dataset. The disclosed systems and methods were used to identify population dynamics based on the first four principal components, and resulting in the same patients being identified to be immune-stable and immune-unstable as when the disclosed systems and methods were run on the entire data set. This indicates that the cell populations present in each principal component not only accurately recapitulate the full dynamics of the individual's immune system, and the same stability and instability of an individual's immune dynamics is also preserved through this transformation. Further, by projecting the large dataset into fewer dimensions, the complex interactions between cells can be better understood.

In some embodiments, using four principal components was the most effective at identifying the relevant immune dynamics, and supplied the most parsimonious models. Table 1 provides a summary of the populations accounting for over eighty percent of the total variance in the dataset corresponding to the eleven patients.

TABLE 2

| PC1 (71% of total variance) | PC2 (8% total variance) | PC3 (7% total variance) | PC4 (3% total variance) |
|---|---|---|---|
| CD3−CD19−: 0.4436 | TotalCD4T-cells: 0.4243 | CD86 + Monocytes: −0.5087 | NKT: 0.4595 |
| CD3+: −0.4301 | CD8T-cells: −0.4152 | ClassicalMonocytes: −0.4080 | T-cells: −0.2985 |
| CD33+: 0.3560 | CD4T-cells: 0.3955 | CD38 + Monocytes: −0.3819 | CS1 + B-cells: 0.2838 |
| T-cells: −0.3438 | GnzB + Monocytes: −0.2881 | Monocytes: −0.3502 | GnzB + Monocytes: −0.2826 |
| TCRa/b: −0.3404 | EMCD8: −0.2795 | T-cells: −0.2538 | TCRa/b: −0.2814 |
| Monocytes: 0.2439 | TIGIT + CD8T-cells: −0.2575 | TCRa/b: −0.2499 | CD3−CD19−: −0.2491 |
| CD38 + Monocytes: 0.1667 | CD38 + CD4T-cells: 0.2061 | CD33+: −0.1783 | TIGIT + NKT-cells: 0.2469 |
| TotalCD4T-cells: −0.1621 | CMCD4: 0.1639 | CD3+: −0.1780 | EMCD8: −0.2015 |
| ClassicalMonocytes: 0.1577 | NaiveCD4: 0.1611 | TotalCD4T-cells: −0.1331 | EMCD4: 0.1804 |
| CD4T-cells: −0.1533 | TemraCD8: −0.1579 | CD4T-cells: −0.1138 | TIGIT + CD8T-cells: −0.1662 |
| CD8T-cells: −0.1424 | CD3−CD19−: 0.1476 | CD3−CD19−: 0.1129 | CD3+: 0.1610 |
| CD86 + Monocytes: 0.1365 | CS1 + NKT-cells: −0.1235 | CD163 + Monocytes: −0.1032 | CMCD4: −0.1534 |
| GnzB + Monocytes: 0.0869 | NKT: −0.1121 | TIM-3 + Monocytes: −0.0927 | ClassicalMonocytes: 0.1500 |
| NKT: −0.0863 | ClassicalMonocytes: −0.1094 | NKT: 0.0759 | TIM-3 + Monocytes: 0.1417 |
| EMCD8: −0.0819 | TIGIT + NKT-cells: −0.1067 | CD8T-cells: −0.0697 | CD86 + Monocytes: 0.1294 |
| EMCD4: −0.0793 | CD69 + CD4T-cells: 0.0897 | CD69 + Monocytes: −0.0654 | CD38 + CD4T-cells: −0.1239 |
| TIGIT + CD8T− cells: −0.0784 | CD86 + Monocytes: −0.0886 | TemraCD8: −0.0619 | CD8T-cells: −0.1158 |
| PD-1 + CD4T-cells: −0.0721 | PD1 + CD8T-cells: −0.0824 | IntermediateMonocytes: −0.0580 | NaiveB-cells: 0.1036 |
| TIGIT + NKT-cells: −0.0545 | B-cells: −0.0721 | NK-cells: 0.0569 | CD38 + CD8T-cells: −0.1031 |
| TemraCD8: −0.0493 | CD3+: −0.0705 | CD38 + NK-cells: 0.0562 | B-cells: 0.1028 |

In some embodiment, variation within the principal component space may be indicative of immune-stable or immune-unstable patient populations. For example, in some embodiments, patients with stable immune systems, who were also clinically stable, tended to have minimal movement within the principal component space. By contrast, in progressing patients identified as immune-unstable by the disclosed systems can demonstrate a large trajectory of movement within principal components within the state space (e.g., PC1 and PC3). This may be indicative of a strong, unstable interactions between these four cell populations represented by these principal components, and may allow researchers to identify methods to stabilize these patients with drugs or optimized dara timing in the future in accordance with the cell populations indicated by these principal components.

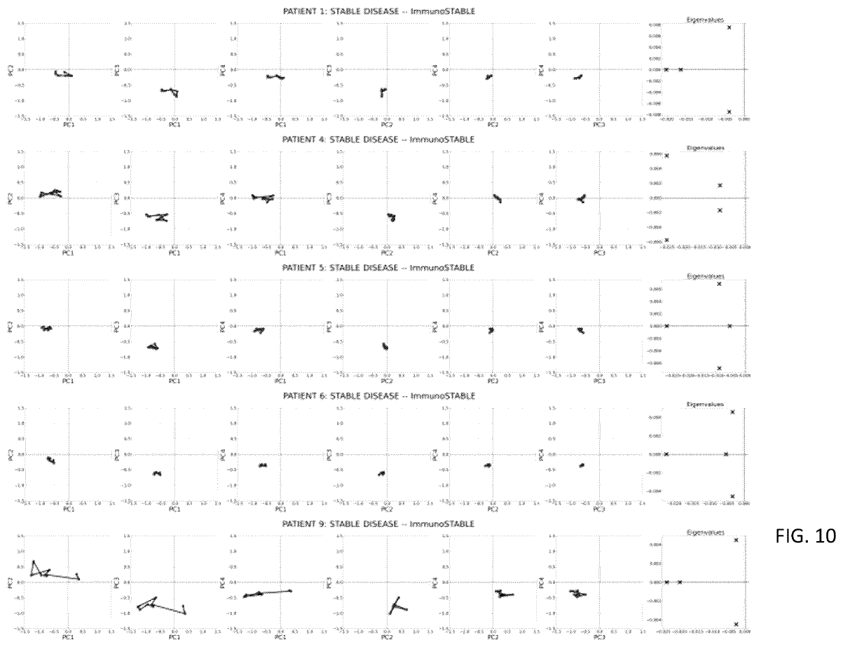

FIG. 10 illustrates the four principal components of five patients identified from the eleven patient experimental data set as being immune-stable. Shown are patients identified as having clinically stable disease while on the trial. Each individual patient is displayed on a row, and the columns are the individual pairings of the principal components. In general, all stable patients displayed relatively little movement in the principal component space, with the exception of the patient in row four. As shown, the eigenvalues for the principal components also illustrate stability.

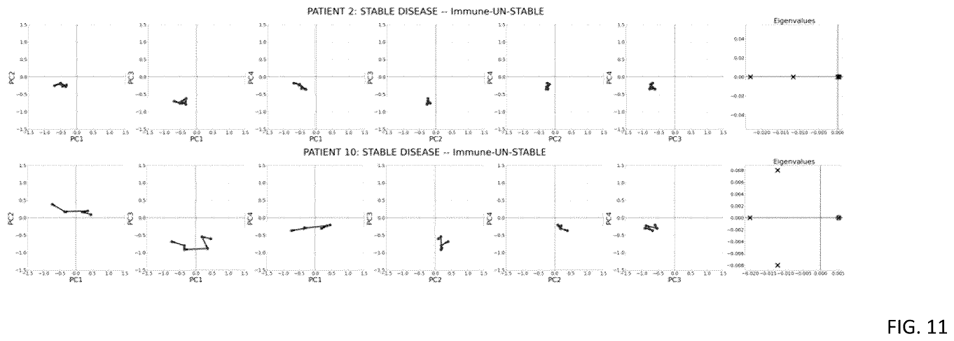

FIG. 11 illustrates the two patients who presented as clinically stable, but were identified as having unstable immune dynamics by the systems and methods described herein. For example, the patient shown in the top row displays a large trajectory in the PC space, while the patient in the lower row appears to have a narrower trajectory.

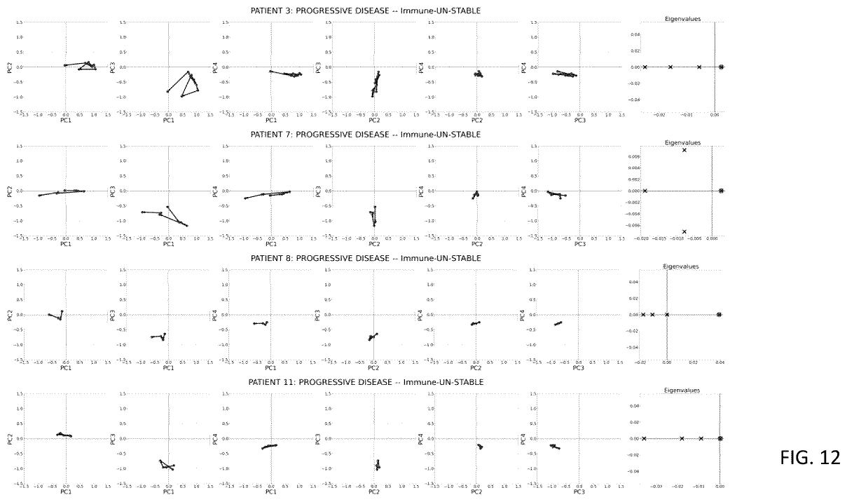

FIG. 12 illustrates patients which progressed in the clinic, and who were identified to be immune-unstable by the systems and methods described herein. In most cases, the trajectory in the principal components space is quite large, and often crosses the axis. In contrast, the immune-stable patients' trajectories often do not cross the axis, and tend to have a narrower, more stable trajectory.

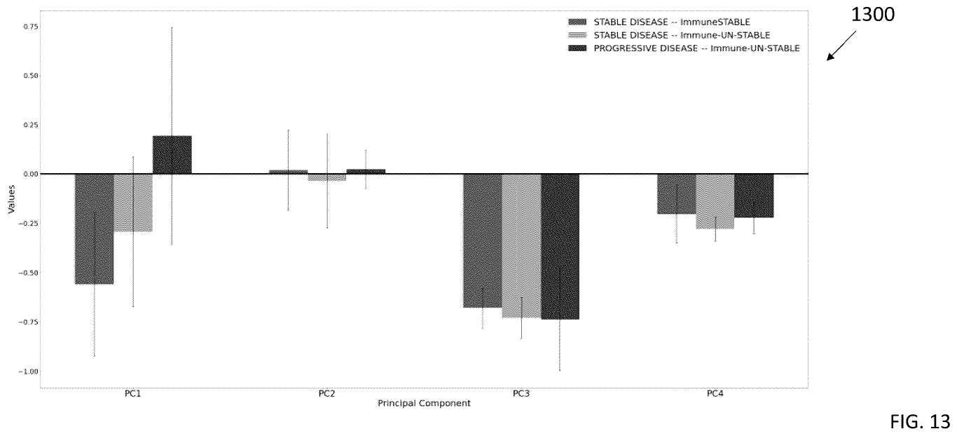

FIG. 13 illustrates the range and variation of each principal component. The bar chart 1300 displays the variation between the populations of patients identified as immune-stable or immune-unstable in accordance with the disclosed systems and methods. In general, patients identified to be immune-stable have lower variation in some principal components (e.g., PC1 and PC3). In comparison, patients who are immune-unstable have larger variation in some principal components (e.g., PC1 and PC3). These results are consistent with the idea that stable trajectories are contained, and vary less than unstable trajectories, which typically have wide variations. Accordingly, the actual dynamic stability or instability in the immune system can be identified using the systems and methods described herein.

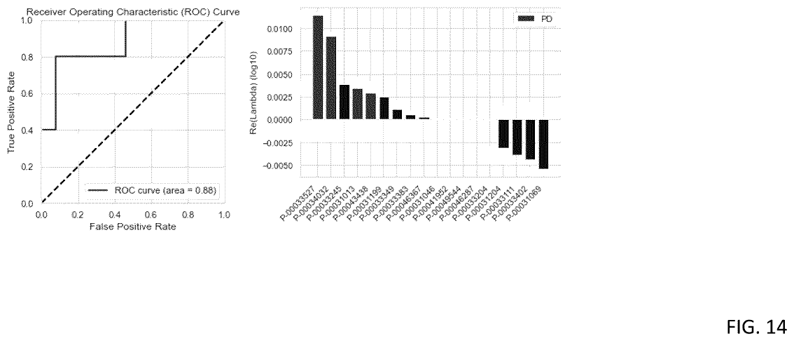

FIG. 14 illustrates experimental results from a set of eighteen patients. Illustrated is a receiver operating characteristic (ROC) curve for a system built in accordance with the description herein applied to predict the progression of multiple myeloma in patients receiving daratumumab (dara) maintenance therapy after autologous stem cell transplant. Also illustrated is a waterfall plot illustrating the real part of the maximum eigenvalue of the patient's individual immune equation, as determined by performing the techniques described herein on time-resolved populations of the populations of interest. Bars on the left indicate patients who progressed on trial. Blue/right bars indicate stable disease, or complete response.

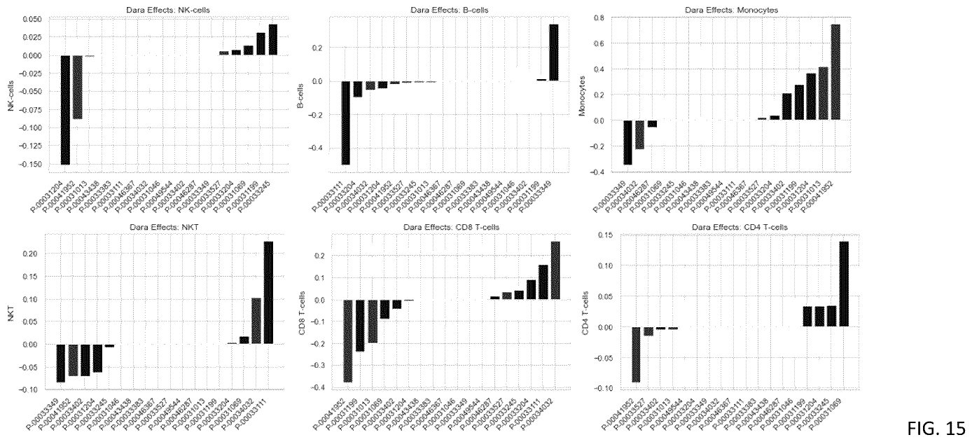

FIG. 15 illustrates the impact of dara on populations of the six cell types identified using experimental data as well as the interactions between these particular cell types. Experimental data included immune cell populations measure from longitudinal peripheral blood samples while on daratumumab maintenance for the following cells: Natural Killer Cells, B-cells, Monocytes, NK T-cells, CD8+ T-cells, an C4+ T-cells). Using a principal components analysis most important cell types contributing toward the stability or instability of the immune system were identified. The resulting subset of cell types were analyzed using their eigenvalues. For example, a coefficient matrix can be determined indicating the impact of one cell type on another cell type. Additionally, the impact of dara on the cell types (B) as well as the impact of one cell type on another cell type (A) can be determined. The eigenvalues can then be used to generate predictions of the stability associated with the immune system. In some aspects, the eigenvalues determined for the experimental data can be used to identify patient specific sensitivities. For example, a patient may be particularly sensitive to losing their B-cell population as shown in the patient's eigenvalues corresponding to the impact of dara on the patient's B-cell population. Accordingly, a treatment plan for the patient can be developed that avoids the use of dara or adjusts the dosage and/or type of medication (e.g., adding/removing additional therapeutic drugs) accordingly.

In one example, the net linear interaction rates of six cell types (Natural Killer cells, B-cells, monocytes, NK T-cells, CD8 T-cells, CD4 T-Cells), along with their interactions with daratumumab can be determined. The changes in each cell population can be modeled as a linear system of ordinary differential equations with unknown coefficients A, and open-loop control matrix B, with control input, u, defined as follows:

$$\dot{x} = Ax + Bu , where ,$$

$$\dot{x} = {[ \dot{NK} , \dot{B} , \dot{Monocytes} , \dot{NKT} , \dot{CD 8 T} , \dot{CD 4 T} , \dot{dara} ]}^{T} ,$$

$$x = {[ N K , B , Monocytes , NKT , CD 8 T , C D 4 T , d a ra ]}^{T} , u = dara ,$$

$$B = [ \begin{matrix}{b}_{d a ra , NK} \\ {b}_{dara , B} \\ {b}_{d a ra , mono} \\ {b}_{d a ra , NKT} \\ {b}_{d a ra , CD 8} \\ {b}_{d a ra , CD 4}\end{matrix} ] ,$$

$$A = [ \begin{matrix}{a}_{NK , NK} & {a}_{B , NK} & {a}_{mono , NK} & {a}_{NKT , NK} & {a}_{CD 8 T , NK} & {a}_{CD 4 T , NK} \\ {a}_{NK , B} & {a}_{B , B} & {a}_{m ono , B} & {a}_{NKT , B} & {a}_{CD 8 T , B} & {a}_{CD 4 T , B} \\ {a}_{NK , mono} & {a}_{B , mono} & {a}_{mono , mono} & {a}_{NKT , mono} & {a}_{CD 8 T , mono} & {a}_{CD 4 T , mono} \\ {a}_{NK , NKT} & {a}_{B , NKT} & {a}_{m ono , NKT} & {a}_{NKT , NKT} & {a}_{CD 8 T , NKT} & {a}_{CD 4 T , NKT} \\ {a}_{NK , CD 8} & {a}_{B , CD 8} & {a}_{mono , CD 8} & {a}_{NKT , CD 8} & {a}_{CD 8 T , CD 8} & {a}_{CD 4 T , CD 8} \\ {a}_{NK , CD 4} & {a}_{B , CD 4} & {a}_{mono , D 4} & {a}_{NKT , CD 4} & {a}_{CD 8 T , CD 4} & {a}_{CD 4 T , CD 4}\end{matrix} ] .$$

The eigenvalues of matrix A determine the innate system stability (Immune stability) and can be predictive of disease progression. Additionally, the controllability matrix, C of an open-loop system is denoted as: C=[BABA²B . . . Aⁿ⁻¹B], where n is the number of species in the system. A system is controllable under controller B, if and only if matrix C is full rank.

Accordingly, in the experimental data with six species in the system (i.e., the six cell types discussed above), the rank of C was less than full rank. Accordingly, the experimental data suggests that no potential dara treatment was sufficient to control the immune system, and as such, the patient's innate immune dynamics alone are likely driving progression while on trial.

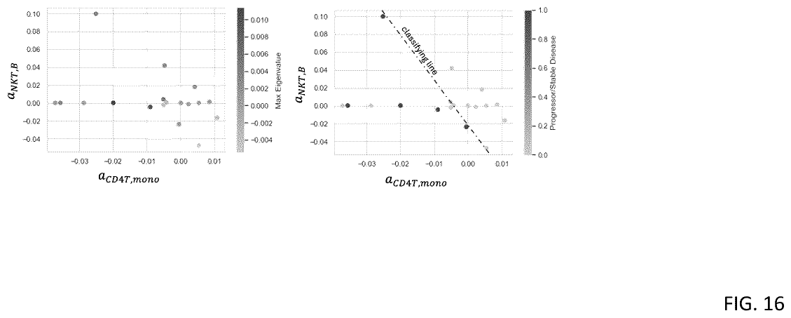

FIG. 16 illustrates experimental results. In some aspects, 42 model coefficients were evaluated to determine the most corelated with immune stability. Experimental data indicated hat coefficients corresponding to interaction between Natural Killer cells and B-cells and CD4 T-cells and monocytes were the coefficients that correlated most strongly with immune stability. In particular α_{NKT,B}, (r=0.32, p=0.19) and α_{CD4T,mono} (r=0.40, p=0.098). In some aspects, a classification on the data illustrated in FIG. 16 can be used to identify patient populations with disease progression and/or unstable immune systems and those without disease progression and/or stable immune systems. In some aspects the classification between the patient populations using a classification line can utilize a receiver operating curve and support vector machine. Illustrated in FIG. 16 are plots of all coefficients α_{CD4T,mono} (x-axis) vs. α_{NKT,B} (y-axis), with data categorized by maximum immune system eigenvalue, with a first group indicating decreased stability, and a second group indicating increased stability. In the right-most plot data is organized by progression status, with a left cluster of dots corresponding to patients who progressed while on dara maintenance, and a right cluster of dots corresponding to patients who remained stable or responded completely while on dara. In some embodiments, the groups can be separated by a classifying line.

### Experiment #2

Chimeric Antigen Receptor (CAR) T-cell therapy has significantly improved clinical outcomes for patients with relapsed or refractory multiple myeloma. Current clinical data demonstrate high overall response rates, ranging from approximately 80% to 100%, and deep initial responses following administration of CAR T cells. Despite these promising results, a substantial proportion of patients experience disease relapse after CAR T-cell therapy. For example, recent long-term follow-up data from the CAR TITUDE-1 study indicate that only about 33% of treated patients remain progression-free at five years post-treatment. While this represents a meaningful advancement in therapeutic efficacy, the majority of patients ultimately exhibit disease progression, underscoring the need for improved strategies to sustain durable responses.

To address this unmet need, the present disclosure provides systems and methods for predicting therapeutic response in patients undergoing B-cell maturation antigen (BCMA)-directed CAR T-cell therapy. The disclosure further enables identification of optimal treatment plans for individuals with relapsed or refractory multiple myeloma, a population for whom therapeutic options remain limited. Given the increasing availability of diverse treatment modalities, it is clinically imperative to determine, prior to initiation, which therapy is most likely to achieve a favorable response in a given patient.

In certain embodiments, the systems and methods describe a system for generating a dynamical model of an immune system configured to analyze and characterize the dynamic immune events occurring during CAR T-cell therapy. This model facilitates early detection of biological markers associated with treatment resistance and disease relapse. By integrating patient-specific data and immune response parameters, the system predicts resistance mechanisms at an early timepoint, thereby enabling timely implementation of mitigation strategies. Such predictive capability improves clinical decision-making, optimizes therapeutic sequencing, and enhances long-term patient outcomes.

The influence of a patient's endogenous immune cell populations on the durability of response to chimeric antigen receptor (CAR) T-cell therapy in multiple myeloma was evaluated. Experimental analyses were conducted to determine whether intrinsic immune cell characteristics impact the duration of disease control following CAR T-cell administration. Comparative profiling revealed distinct immunological patterns between patients exhibiting prolonged therapeutic benefit (“durable responders”) and those with shorter response intervals (“non-durable responders”).

In certain embodiments, durable responders demonstrated significantly greater CAR T-cell expansion and persistence relative to non-durable responders. Additionally, specific subsets of the patient's native immune cells were found to be elevated in durable responders compared to non-durable responders, suggesting a contributory role in sustaining CAR T-cell activity. Computational modeling was employed to identify novel associations between immune cell dynamics and CAR T-cell performance. For example, the stimulatory capacity of B-cell populations on CAR T-cell activation was identified as a critical determinant of therapeutic efficacy. These findings support the development of predictive algorithms and biomarker-based strategies to optimize patient selection and improve long-term outcomes in CAR T-cell therapy.

A clinical study was conducted involving a cohort of twenty-five (25) evaluable patients diagnosed with multiple myeloma and treated with B-cell maturation antigen (BCMA)-directed CAR T-cell therapy. For the purposes of this study, “evaluable patients” were defined as individuals for whom successful sample collection and processing were achieved at baseline and at a minimum of fifty percent (50%) of the scheduled timepoints.

Participants were stratified based on duration of therapeutic response. Twelve (12) patients exhibited a response duration greater than eleven (11) months and were classified as “durable responders.” Thirteen (13) patients exhibited a response duration of eleven (11) months or less and were classified as “non-durable responders.” Across all participants, a total of one hundred and nine (109) peripheral blood samples were collected for subsequent immunological and computational analyses.

Immune correlates of response durability were investigated in patients with relapsed or refractory multiple myeloma treated with B-cell maturation antigen (BCMA)-directed CAR T-cell therapy. High-dimensional multiparameter flow cytometry (CyTEK platform) was employed to longitudinally profile BCMA CAR T cells in conjunction with a comprehensive set of immune cell populations. These included adaptive immune compartments (T cells and B cells) and innate immune compartments (natural killer (NK) cells, natural killer T (NKT) cells, monocytes, and myeloid-derived populations), assessed across multiple post-infusion timepoints.

Flow cytometric analysis utilized a 28-marker panel comprising 27 immune phenotyping markers and one BCMA CAR-specific detection marker for quantification of CAR-expressing T cells. This panel enabled detailed characterization of cellular activation states, exhaustion phenotypes, memory formation, and accessory cell dynamics. The objective of this analysis was to identify phenotypic and functional features distinguishing long-term responders from patients exhibiting early relapse, thereby providing cellular-resolution insights into immune dynamics following CAR T-cell infusion.

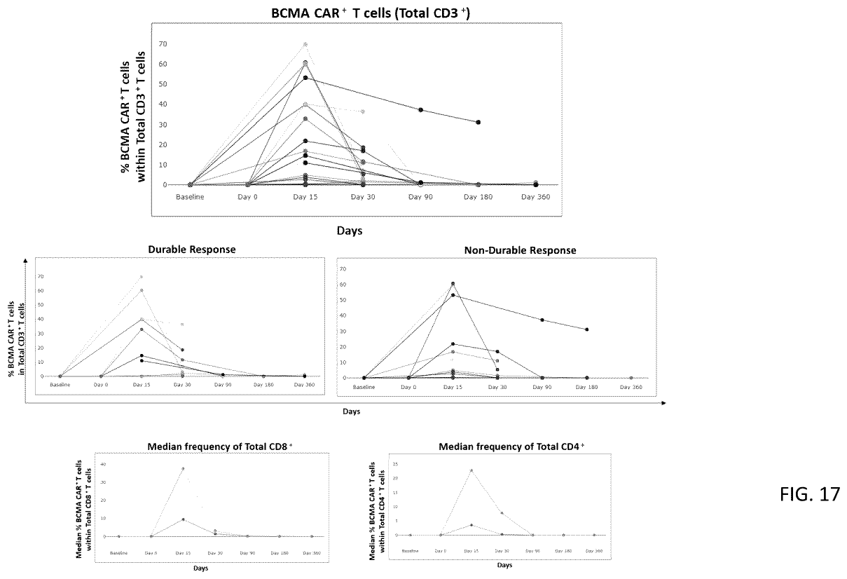

Longitudinal analysis of BCMA-directed CAR T-cell kinetics was performed across all study participants. As illustrated in FIG. 17, quantification of CAR T-cell expansion revealed that peak proliferation occurred at approximately day fifteen (15) post-infusion in all subjects. Comparative assessment demonstrated that the magnitude of peak expansion was significantly greater in durable responders (response duration >11 months) relative to non-durable responders (response duration≤11 months). Further phenotypic characterization indicated that CD8⁺ CAR T-cell expansion exceeded that of CD4⁺ CAR T cells during the peak proliferative phase (see FIG. 1). Persistence of CAR T cells was evaluated using an area-under-the-curve (AUC) metric across the observation period. No statistically significant differences in CAR T-cell persistence were observed between durable and non-durable responder cohorts.

FIG. 17 illustrates that BCMA CAR T cell expansion peaks at Day 15 across all patients, separated by those experiencing a durable response and those experiencing a non-durable response, and that CD8⁺ T cells show greater peak expansion than CD4⁺ T cells.

immune profiling was performed to evaluate cellular correlates of response durability following BCMA-directed CAR T-cell therapy. High-dimensional flow cytometry analysis revealed consistent immunological signatures within innate compartments, particularly natural killer (NK) and natural killer T (NKT) cell subsets, associated with durable clinical response (defined as >11 months).

Baseline profiling indicated that NK subsets expressing CD38⁺, HLA-DR⁺, CD27⁺, CD25⁺, CD69⁺, and TIM-3⁺ were elevated in non-durable responders but subsequently recovered and became enriched in durable responders at later timepoints. Conversely, NKT subsets characterized by CD25⁺, CD38⁺, TIGIT, and TIM-3⁺ expression were significantly elevated at baseline in durable responders and remained relatively stable post-infusion, with enriched expression patterns persisting in this cohort. Monocyte activation markers (e.g., CD69⁺) and classical monocyte phenotypes aligned with these findings, supporting a systems-level innate immune readiness associated with durable response.

Longitudinal CyTEK data demonstrated convergence of NK/NKT differences by Days 90-180, suggesting temporal immune remodeling. Naïve T-cell pools (CD4⁺ and CD8⁺) were better preserved in durable responders, indicating superior immune fitness. Activated NK cells, activated T cells, and central memory T cells were consistently elevated in durable responders. CD8⁺ T-cell activation was observed from Day 0 through Day 180 and persisted from Day 30 to Day 360. CD4⁺ T helper cells expressing CD69⁺ were elevated at Days 15 and 30, while CD8⁺CD69⁺ cells were enriched at Days 30 and 180.

Monocytic myeloid-derived suppressor cells (MDSCs) exhibited reduced suppressive activity in durable responders at Days 15 and 90, whereas non-durable responders demonstrated immune suppression, reinforcing that durability of response is multifactorial and partly driven by innate immune mechanisms. The CD4/CD8 ratio showed no statistically significant differences across timepoints (p>0.2), although a positive trend was observed in durable responders.

Among patients achieving complete response (CR), total CD3⁺ and CD8⁺ T-cell counts were higher compared to those with partial response (PR) or progressive disease (PD), whereas CD4⁺ T-cell counts did not differ significantly. Naïve B cells, NKT cells (including TIGIT and TIM-3⁺ subsets), and TCRγδ populations were also elevated in patients achieving CR.

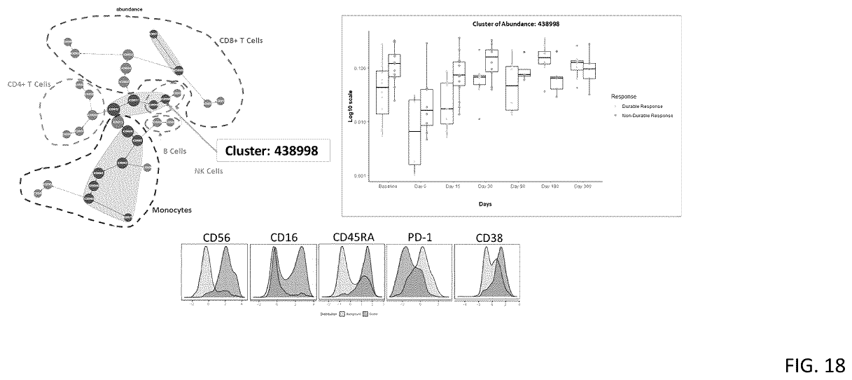

As shown in FIG. 18 exploratory computational analysis was performed using the CITRUS algorithm to identify immune cell clusters associated with response durability following BCMA-directed CAR T-cell therapy. This analysis revealed the emergence of a distinct NK-like population at later timepoints in durable responders. The identified cluster was characterized by the expression profile CD38⁺ CD16⁺ CD45⁺ RA CD56⁺ PD-1⁻, indicative of an activated NK phenotype with absence of PD-1 expression. The lack of PD-1 suggests reduced engagement of inhibitory checkpoint pathways, potentially contributing to sustained immune activity and prolonged therapeutic response. These findings highlight a novel NK-like signature that may serve as a biomarker for durable clinical benefit and inform strategies to enhance CAR T-cell efficacy. These trends may inform dynamic modeling of resistance mechanisms.

The findings described herein demonstrate dynamic immune interactions that differentiate durable responders from non-durable responders following BCMA-directed CAR T-cell therapy. Durable responders exhibited elevated expression of activation, co-stimulatory, and memory-associated markers in CAR⁺ T cells, accompanied by supportive innate immune signals and reduced expression of inhibitory receptors. These features collectively suggest mechanisms contributing to CAR T-cell persistence and enhanced immune fitness. Furthermore, the identification of a late-emerging NK-like population characterized by a checkpoint-negative phenotype (PD-1) indicates reduced checkpoint engagement, which may facilitate sustained CAR T-cell activity. Collectively, these observations reveal that CAR T-cell persistence is not exclusively intrinsic to T cells but is influenced by innate immune remodeling and the presence of checkpoint-negative NK-like subsets, representing a previously unrecognized mechanism underlying durable clinical response.

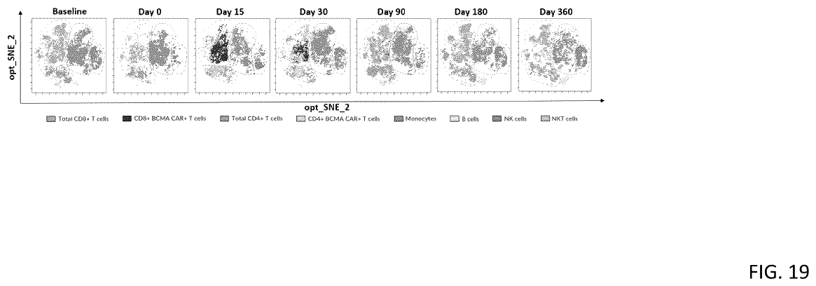

FIG. 19 illustrates representative t-SNE plots illustrating longitudinal distribution and remodeling of major immune cell populations. This includes CD4⁺ and CD8⁺ T cells (with BCMA CAR⁺ subsets), monocytes, B cells, NK cells, and NKT cells from baseline through Day 360 post-infusion. Clustering patterns demonstrate dynamic immune shifts over time.

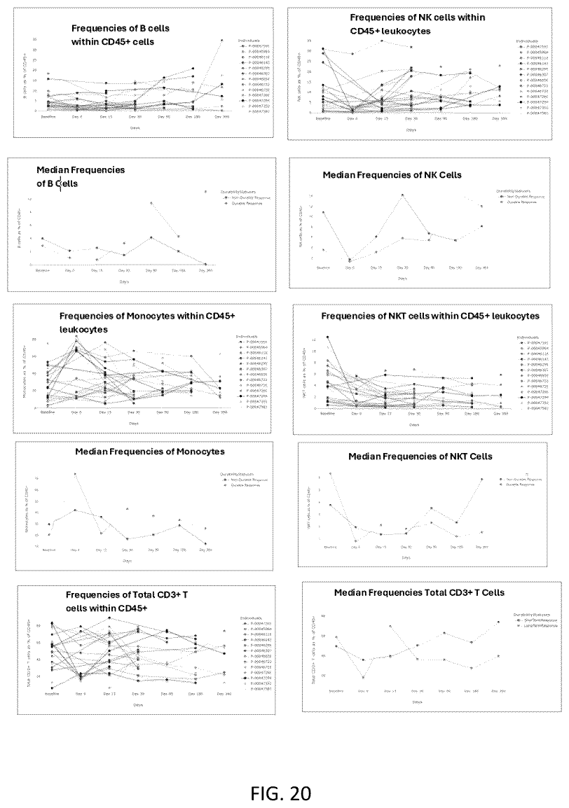

FIG. 20 illustrates longitudinal dynamics of immune cell subsets, including frequencies of B-cells within CD45+ cells, frequencies of NK cells within CD45+ leukocytes, median frequencies of B-Cells, median frequencies of NK cells, frequencies of monocytes within CD45+ leukocytes, frequencies of NKT cells within CD45+ leukocytes, median frequencies of mnoocytes, median frequencies of NKT cells, frequencies of Total CD3+ T-cells within CD45+, and median frequencies of total CD3+ T-cells.

### Experimental Methods: Modeling Criteria

To enable mathematical modeling of immune dynamics, a minimum of two CyTeK-based measurements were obtained following administration of chimeric antigen receptor (CAR) T-cell therapy. In a representative study cohort comprising twenty-nine patients, twenty-six patients provided at least two post-infusion measurements, while sixteen patients provided three or more measurements, thereby ensuring sufficient longitudinal data for computational analysis and model generation.

### Experimental Methods: Cell Populations

Because the average number of post-infusion measurements following CAR T-cell administration was approximately three samples and the CyTEK platform quantified over one hundred distinct immune cell populations, the dimensionality of the dataset was reduced for computational efficiency and to mitigate overfitting. Accordingly, six representative immune cell populations, along with CAR T cells, were selected for mathematical modeling as summarized in Table 3. This selection enabled generation of a parsimonious model while preserving the most relevant biological interactions for analysis.

TABLE 3
Populations used in dynamic mathematical analysis

| Population Index | Cell Population |
|---|---|
| 1 | NK cells |
| 2 | NKT cells |
| 3 | B cells |
| 4 | Monocytes |
| 5 | CD8+ T cells |
| 6 | CD4+ T cells |
| 7 | CAR T cells |

### Experimental Methods: Modeling

In some implementations, a physics-informed data science algorithm that aims to learn mathematical equations directly from the data was utilized. In contrast to conventional model-data regression where a model is specified a priori and subsequently fit to the data by minimizing the model-data error, the Sparse Identification of Nonlinear Dynamics (SINDy, Brunton et al. PNAS 2016) algorithm performs the regression across a family of functions to identify the most parsimonious model. The SINDy algorithm has several advantages, most notably, that it enables data-driven modeling that both ‘fits the data’ and also identifies families of models that can be compared and analyzed for mechanistic insights and interpretability. The output of the SINDy algorithm is a set of parameterized equations best describing the data, under pre-specified simplified assumptions. Briefly, SINDy assumes that the rate of change of an observable quantity, \dot{y}, can be estimated by regression of the state function, y, other relevant measurables (Θ), and the coefficient matrix, E.

$$\dot{y} = Θ ( y ) Ξ \qquad ( 1 )$$

In some implementations, the state function is the relative proportion of the 6 immune cell populations and CAR T-cells given by CyTek analysis. SINDy was performed on longitudinal samples collected from each patient, using the relative proportion of NK cells, NKT cells, B cells, monocytes, CD8+ and CD4+ T-cells, and CAR T cells. Temporal derivatives were approximated using a first order forward finite difference estimation method, and the library for each term consisted of linear interaction terms for each immune cell population and CAR T− cells. The SINDy regression equation is solved and coefficients E, denoting the interactions of each species are estimated:

$$Ξ = [ \begin{matrix}{a}_{1 , 1} & {a}_{1 , 2} & … & {a}_{1 , 7} \\ {a}_{2 , 1} & {a}_{2 , 2} & … & {a}_{2 , 7} \\ ⋮ & ⋮ & ⋱ & ⋮ \\ {a}_{7 , 1} & {a}_{7 , 2} & … & {a}_{7 , 7}\end{matrix} ] , Ξ ∈ {ℝ}^{7 \times 7} . \qquad ( 2 )$$

The matrix Θ is comprised of the individual species at each measurement,

$$Θ = [ \begin{matrix}│ & │ & & │ \\ {y}_{1} & {y}_{2} & … & {y}_{7} \\ │ & │ & & │\end{matrix} ] , Θ ∈ {ℝ}^{n \times 7} , \qquad ( 3 )$$

where n is the number of time samples after truncation to match \dot{y}, the rate of change measured in each population.

$$\dot{y} = [ \begin{matrix}│ & │ & & │ \\ {\dot{y}}_{1} & {\dot{y}}_{2} & … & {\dot{y}}_{\dot{7}} \\ │ & │ & & │\end{matrix} ] , Θ ∈ {ℝ}^{n \times 7} . \qquad ( 4 )$$

As there are 7 interacting species, the L1 Lasso objective function, with a sparsity-enforcing parameter λ=1.0E−6, was utilized to limit the interactions identified. Interactions between cell populations X and Y as “The effect of X on Y” is noted in shorthand as “Y←X”.

### Experimental Results

A dynamical model for immune stability was constructed to characterize interactions between immune cell populations and BCMA-directed CAR T cells. The model was fitted for each patient using the Sparse Identification of Nonlinear Dynamics (SINDy) method, enabling identification of 49 interaction coefficients representing immune system dynamics. Eigenvalues (λ) of the coefficient matrix (Ξ) were computed to provide a quantitative measure of immune interactivity and system stability for each patient. Using the identified model and corresponding coefficients, forward simulations were performed to predict immune trajectories over time. These simulated trajectories were then compared with actual patient data and the associated eigenvalue spectrum to validate model accuracy and assess dynamic behavior, including oscillatory patterns and stability characteristics.

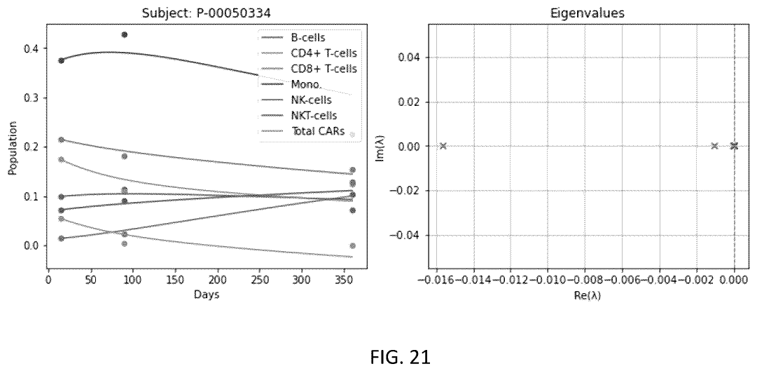

FIG. 21 illustrates experimental results for one patient. The left panel of FIG. 21 illustrates immune cell populations measured by high-dimensional flow cytometry (CyTek) shown as discrete data points, overlaid with the solution generated by the identified immune mathematical model represented by solid lines. This comparison illustrates the model's ability to capture observed immune dynamics over time. The right panel of FIG. 21 illustrates real and imaginary components of the eigenvalue (λ) spectrum derived from the coefficient matrix (Ξ) associated with the model. Eigenvalues with non-zero imaginary components indicate oscillatory behavior within the immune system, while real components reflect growth or decay dynamics. A positive real component in any eigenvalue denotes mathematical instability of the system. In the example shown, all eigenvalues exhibit non-positive real components, predicting stable immune dynamics for the subject.

Analysis of immune interaction dynamics revealed significant correlations between progression-free survival (PFS) and specific cell-to-cell interaction coefficients derived from the immune stability model generated in accordance with the methods described herein. Notably, PFS was strongly associated with CD4⁺ T-cell interactions with CD8⁺ T cells (R=−0.82, p=3.8×10⁻³) and CAR T cells (R=−0.69, p=2.6×10⁻²), as well as CD8⁺ T-cell interactions with B cells (R=0.68, p=3.0×10⁻²). Overall survival demonstrated a positive correlation with CD8⁺ T-cell interactions with B cells (R=0.71, p=2.2×10⁻²). Statistically significant correlations among dynamical rate coefficients were further analyzed using hierarchical clustering to identify co-correlated immune populations and interaction patterns within the CAR T-treated cohort.

To assess immune system stability, the eigenvalue spectrum of the identified immune stability model was employed as a proxy metric. Interactions between CAR T cells and monocytes were positively correlated with instability (monocytes←CARs: R=0.86, p=1.3×10⁻³), suggesting that CAR-mediated stimulation of myeloid populations may contribute to treatment failure and reduced immune stability. Conversely, immune stability was negatively correlated with CD8⁺ T-cell stimulation by CAR T cells (CD8⁺ T cells←CARs: R=−0.87, p=9.9×10⁻⁴), indicating that CAR-driven activation of CD8⁺ T cells promotes a more stable immune profile and improved therapeutic response. Additionally, interactions between CAR T cells and B cells (CARs←B cells: R=−0.94, p=7.0×10⁻⁵) were strongly associated with enhanced immune stability, suggesting that B-cell-mediated stimulation of CAR T-cell proliferation is a key determinant of durable immune dynamics and clinical benefit.

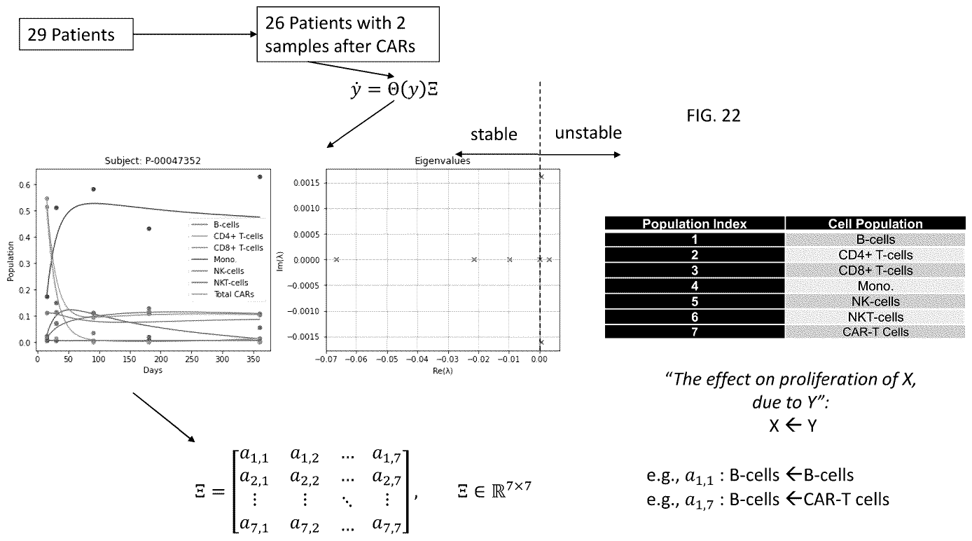

FIG. 22 illustrates a summary of the experiments performed. A total of 29 patients having longitudinal data receiving CAR T-cell therapy were studied. An immune stability model was generated for each patient and coefficients corresponding to the immune stability model were solved for each patient's test data. The eigenspace of each immune stability model was determined and eigenvalues were evaluated as to whether they were above or below a threshold to determine if the eigenvalues corresponded to stable or unstable immune states. Coefficients of the immune stability model were indicative of the effect of proliferation of cell populations on each other.

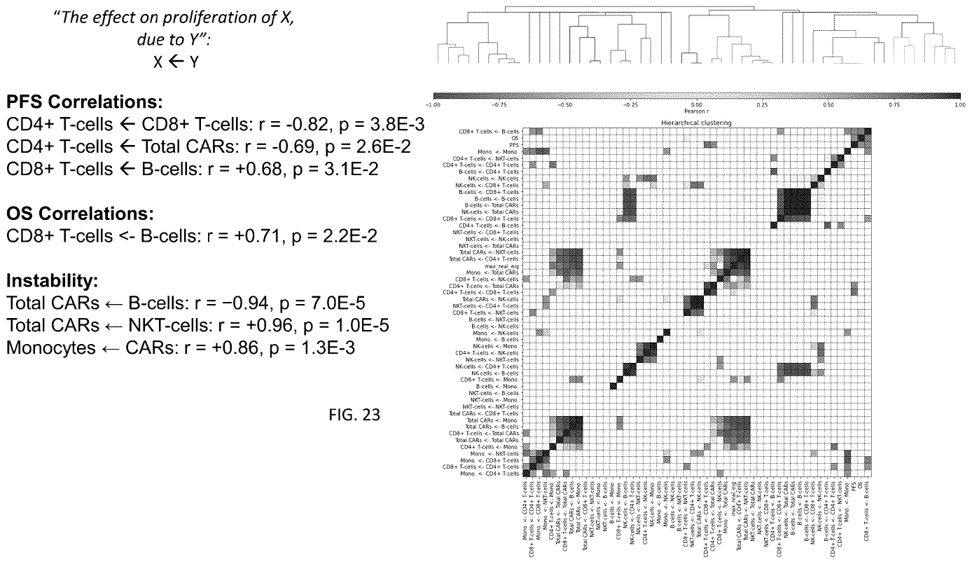

FIG. 23 illustrates results and interactions between CAR T Cells and monocytes including correlations related to progression-free survival (PFS), overall survival (OS), and/or instability. A top panel presents a dendrogram generated from hierarchical clustering of population interaction rates alongside clinical outcomes, including overall survival (OS) and progression-free survival (PFS). The right side panel displays a correlation clustergram illustrating relationships between cell-to-cell interaction coefficients and survival metrics. Grid elements are coded based on statistical significance of Pearson correlation values: solid colors indicate p<0.05, semi-transparent colors represent p-values between 0.05 and 0.1, and empty cells denote non-significant correlations. This visualization highlights co-correlated immune interactions and their association with clinical outcomes in CAR T-treated patients.

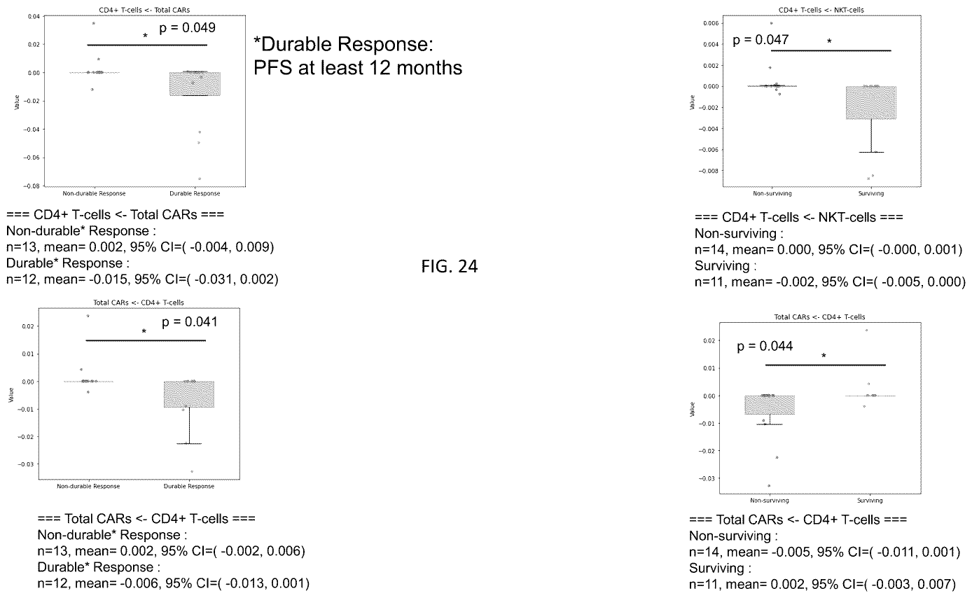

FIG. 24 illustrates categorizing patient outcomes using binary cutoffs rather than continuous measures of overall survival (OS) or progression-free survival (PFS). Patients are grouped based on whether they survive or progress beyond one year, defining a “durable response.” Predictive interactions identified include B cells→CAR T cells and CD4⁺ T cells→CAR T cells, which correlate with progression beyond one year. No specific relationship with immune stability was observed in this categorical analysis.

### Additional Experimental Results

Analysis of CAR T-cell activation and expansion revealed marked differences between durable responders (defined as progression-free survival >11 months) and non-durable responders (≤11 months). Both CAR CD4⁺; and CAR CD8⁺ T-cell subsets exhibited robust expansion in durable responders compared to non-durable responders. Specifically, CD8⁺ BCMA CAR⁺ T cells peaked at approximately 38% of total CD8⁺ T cells in durable responders versus 9% in non-durable responders at Day 15, representing a four-fold higher peak expansion. From Day 0 to Day 15, durable responders demonstrated an approximate 38-fold increase in CD8⁺ CAR T cells compared to a nine-fold increase in non-durable responders. Both cohorts exhibited contraction by Day 30, with levels declining to approximately 4% in durable responders and 2% in non-durable responders. Similarly, CD4⁺ BCMA CAR T cells peaked at approximately 22% of total CD4⁺ T cells in durable responders versus 4% in non-durable responders (a 5.5-fold difference), with expansion from Day 0 to Day 15 reaching 22-fold in durable responders compared to four-fold in non-durable responders. By Day 30, CD4⁺ CAR T-cell levels declined to approximately 8% in durable responders and were nearly undetectable in non-durable responders. Furthermore, activation markers including CD69, CD25, CD38, and HLA-DR were highly enriched in CD4⁺ CAR T cells of durable responders at Day 15, with further increases observed at Day 30. Notably, HLA-DR expression demonstrated statistically significant elevation at Day 30 in durable responders, indicating sustained activation and immune fitness associated with prolonged therapeutic response.

Analysis of checkpoint marker expression revealed that PD-1, TIM-3, LAG-3, TIGIT, and CTLA-4 were minimally detected at later timepoints (Day 90-180), which is consistent with reduced persistence of CAR T cells during this phase. When these inhibitory receptors were present at sustained levels, their expression correlated with CAR T-cell dysfunction and phenotypic features of T-cell exhaustion. Such persistent checkpoint engagement was associated with diminished therapeutic efficacy and contributed to non-durable clinical responses, indicating that checkpoint marker profiling may serve as a predictive indicator of CAR T-cell performance and long-term treatment outcomes.

Co-stimulatory and survival-associated markers were evaluated in CD4⁺ CAR⁺ T-cell populations following BCMA-directed CAR T-cell infusion. Analysis revealed that CD28, CD27, and CD127 (IL-7Rα) were highly enriched in CD4⁺ CAR⁺ T cells, with expression peaking at Day 15 and remaining elevated through Day 30. Notably, IL-7Rα demonstrated statistically significant elevation at Day 30 in durable responders, indicating enhanced survival signaling and memory formation within this subset. These findings suggest that co-stimulatory and cytokine receptor pathways contribute to prolonged CAR T-cell persistence and improved clinical outcomes.

Memory and differentiation markers were analyzed to assess the preservation of naïve and central memory (CM) T-cell pools in patients treated with BCMA-directed CAR T-cell therapy. Expression of CD45RA and CCR7 revealed that durable responders exhibited significant enrichment of naïve T-cell subsets over time. Specifically, naïve CD8⁺ T cells were significantly enriched at Day 90, while naïve CD4⁺ T cells demonstrated significant enrichment at both Day 30 and Day 90. Additionally, central memory CD4⁺ BCMA CAR⁺ T cells and central memory CD8⁺ BCMA CAR⁺ T cells showed increased representation at Day 15, indicating early memory formation within CAR T-cell populations. These findings suggest that preservation of naïve and CM pools contributes to immune fitness and durability of response.

Innate and accessory immune cell dynamics were evaluated to determine their association with response durability following BCMA-directed CAR T-cell therapy. Analysis revealed that activated NK and NKT cells, as well as CD69⁺ monocytes, were significantly elevated in durable responders at Day 15, with NKT cells already demonstrating a statistically significant increase at baseline in this cohort. Classical monocyte markers, including CD14, CD16, and HLA-DR, were enriched in durable responders, indicating enhanced monocyte activation. NK and NKT cells exhibited higher baseline and early post-infusion levels in non-durable responders; however, durable responders showed delayed but sustained recovery of these populations over time. Additionally, monocytic myeloid-derived suppressor cells (MDSCs) displayed reduced suppressive activity in durable responders at Days 15 and 90, suggesting a favorable innate immune environment. Exploratory CITRUS analysis identified a late-emerging NK-like cluster characterized by CD38⁺CD16⁺CD45⁺RA⁺CD56⁺PD-1⁻ expression in durable responders, indicative of reduced checkpoint engagement and supporting prolonged CAR T-cell persistence. These findings highlight the role of innate immune remodeling and checkpoint-negative NK-like populations in mediating durable clinical responses.

Monocyte dynamics were evaluated to assess their role in immune modulation following BCMA-directed CAR T-cell therapy. Monocyte levels increased sharply at Day 0, reflecting lymphodepletion-induced immune activation prior to CAR T-cell infusion. By Day 15, monocyte counts declined substantially as CAR T cells expanded and the immune system transitioned toward an adaptive response. Notably, durable responders maintained consistently higher monocyte levels compared to non-durable responders through Day 90, suggesting that sustained innate immune support contributes to CAR T-cell persistence and improved therapeutic durability.

B-cell dynamics were evaluated longitudinally to assess their association with response durability following BCMA-directed CAR T-cell therapy. At baseline, both durable responders (progression-free survival >11 months) and non-durable responders (≤11 months) exhibited low B-cell percentages, approximately 5-10%. A sharp decline in B-cell levels was observed at Day 0, consistent with lymphodepletion prior to CAR T-cell infusion. From Day 0 to Day 15, B-cell counts continued to decrease, reflecting BCMA CAR T-cell targeting of plasma cells and mature B cells. Durable responders demonstrated a pronounced and sustained recovery of B-cell levels beginning at Day 30 and continuing through Day 360, with the highest levels observed at the end of follow-up. In contrast, non-durable responders exhibited slower and less robust recovery, with B-cell percentages remaining consistently lower than those of durable responders across all subsequent timepoints. These findings suggest that early and sustained B-cell reconstitution may serve as a biomarker for durable clinical response.

Expression of B and T lymphocyte attenuator (BTLA, CD272) was evaluated across multiple T-cell subsets following BCMA-directed CAR T-cell therapy. BTLA expression was consistently lower in CD4⁺ T cells of durable responders compared to non-durable responders across all timepoints, with statistically significant reduction observed at Day 15. This trend was mirrored in CD4⁺ BCMA CAR⁺ T cells from Day 15 through Day 90, indicating sustained downregulation of BTLA in this subset. Additionally, CD8⁺ BCMA CAR⁺ T cells exhibited lower BTLA expression in durable responders at Days 90 and 180. These findings suggest that reduced BTLA expression may contribute to enhanced CAR T-cell activation and persistence, supporting improved clinical outcomes.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A computer-implemented method, comprising:
generating, based at least on time series data of a quantity of one or more markers present in a blood sample of a subject, a dynamical system model representative of an immune system of the subject, the dynamical system model including one or more functions corresponding to a derivative of the time series data, and the generating of the dynamical system model includes performing a regression to identify, within a library of candidate functions, the one or more functions for inclusion in the dynamical system model;
determining a steady state solution for the one or more functions included in the dynamical system model; and
determining, based at least on the steady state solution for the one or more functions included in the dynamical system model, a stability of the immune system of the subject.

2. The method of claim 1, wherein the subject is a patient with multiple myeloma.

3. The method of claim 1, wherein the subject is a healthy individual.

4. The method of claim 1, wherein the subject is a patient with an immune mediated cancer.

5. The method of claim 1, wherein the regression is a sparse regression.

6. The method of claim 1, wherein the time series data is derived from at least one of data from blood analysis, the blood analysis comprising: complete blood count, differential white blood cell count, spectral flow cytometry (Cytek), or Mass cytometry (CyTOF).

7. The method of claim 5, wherein the sparse regression is performed to determine a sparse vector of coefficients indicating the one or more functions as being active.

8. The method of claim 1, wherein the library of candidate functions include one or more constant functions, polynomial functions, and trigonometric functions.

9. The method of claim 1, wherein the one or more functions comprise nonlinear functions.

10. The method of claim 1, wherein the steady state solution of the one or more functions included in the dynamical system model includes one or more eigenvalues.

11. The method of claim 10, wherein the immune system of the subject is determined to be stable based at least on the one or more eigenvalues not exceeding a threshold value, and wherein the immune system of the subject is determined to be unstable based at least on the one or more eigenvalues exceeding the threshold value.

12. The method of claim 11, wherein the threshold value is zero.

13. The method of claim 1, wherein the derivative of the time series data comprises a numerical approximation and the derivative of the time series data is measured.

14. (canceled)

15. The method of claim 1, wherein the time series data of the quantity of one or more markers present in the blood sample of the subject comprises a quantity of peripheral blood mononuclear (PBMCs) present in the blood sample, or quantity of materials released by tumor cells.

16. The method of claim 1, further comprising:
performing, on the blood sample of the subject, a cytometry by time of flight (CyTOF) to determine the quantity of the one or more markers present therein or performing, on the blood sample of the subject, spectral cytometry (CyTEK) to determine the quantity of the one or more markers present therein.

17. (canceled)

18. The method of claim 1, wherein one or more antibodies are used to determine the quantity of one or more markers present in the blood sample, the one or more antibodies including ANTI-FITC (CD38 multi-epitope), CD86/B7.2, CD45, CD80/B7-1, CD127/IL.7Ra, CD27, CD3, CD19, CD69, CD138/Syndecan 1, CD25, CD64, CD33, GRANZYME B, CD15/SSEA-1, CD8, HLA-DR, CD45RA, CD279/PD-1, CD14, CD56, CD223/LAG-3, TIGIT, CD269 (BCMA), CD319 (CS-1), gammadelta2 (TCR Vdelta2), CD163, TIM-3, CD16, CD4, CD274/PDL1, CD197/CCR7, CD11b, IgG-Kappa, IgG-Lambda, M-protein, and/or CD20.

19. The method of claim 1, further comprising:
determining, based at least on the stability of the immune system of the subject or one or more identified cellular interaction terms, one or more of (i) a first likelihood of the subject responding to a treatment, (ii) a second likelihood of the subject relapsing after the treatment, and (iii) a durability of the subject's response to the treatment.

20. The method of claim 19, wherein the treatment affects the immune system of the subject, and the treatment includes one of Daratumumab and Chimeric Antigen Receptor (CAR) T-cell therapy.

21-22. (canceled)

23. The method of claim 1, further comprising:
identifying, based at least on the stability of the immune system of the subject, one or more treatments affecting the immune system of the subject.

24. The method of claim 1, further comprising:
determining, based at least on the stability of the immune system of the subject, one or more of a dosage and a timing of one or more treatments affecting the immune system of the subject.

25. The method of claim 1, wherein the dynamical system model of the immune system of the subject is further determined based at least on a quantity of circulating myeloma plasma cells present in the blood sample of the subject.

26. The method of claim 25, further comprising:
determining, based at least on the quantity of circulating myeloma plasma cells present in the blood sample of the subject, a level of minimal residual disease for the subject.

27. The method of claim 25, further comprising:
determining, based at least on the stability of the immune system of the subject, a level of minimal residual disease for the subject.

28. The method of claim 1, wherein the dynamical system model representative of the immune system of the subject is further generated based time series data of a level of minimal residual disease present in the subject with an immune-mediated cancer.

29. The method of claim 1, wherein generating of the dynamical system model includes filtering the library of candidate functions to exclude functions within the library of candidate functions having an oscillation frequency exceeding two times a maximum sampling frequency of the data.

30. The method of claim 1, further comprising: reducing a dimensionality of the time series data by applying principal components analysis.

31. A system, comprising:
at least one data processor; and
at least one memory storing instructions, which when executed by the at least one data processor, result in operations comprising:
generating, based at least on time series data of a quantity of one or more markers present in a blood sample of a subject, a dynamical system model representative of an immune system of the subject, the dynamical system model including one or more functions corresponding to a derivative of the time series data, and the generating of the dynamical system model includes performing a regression to identify, within a library of candidate functions, the one or more functions for inclusion in the dynamical system model;
determining a steady state solution for the one or more functions included in the dynamical system model; and
determining, based at least on the steady state solution for the one or more functions included in the dynamical system model, a stability of the immune system of the subject.

32. A non-transitory computer readable medium storing instructions, which when executed by at least one data processor, result in operations comprising:
generating, based at least on time series data of a quantity of one or more markers present in a blood sample of a subject, a dynamical system model representative of an immune system of the subject, the dynamical system model including one or more functions corresponding to a derivative of the time series data, and the generating of the dynamical system model includes performing a regression to identify, within a library of candidate functions, the one or more functions for inclusion in the dynamical system model;
determining a steady state solution for the one or more functions included in the dynamical system model; and
determining, based at least on the steady state solution for the one or more functions included in the dynamical system model, a stability of the immune system of the subject.

33-38. (canceled)
